(19) United States
(12) Patent Application Publication
(10) Pub. No.: US 2014/0371094 A1
(43) Pub. Date: Dec. 18, 2014

(54) METHODS FOR TREATING, DIAGNOSING AND MONITORING ALZHEIMER'S DISEASE

(71) Applicant: F. HOFFMAN-LA ROCHE AG, BASEL (CH)

(72) Inventors: Timothy W. Behrens, South San Francisco, CA (US); Robert R. Graham, South San Francisco, CA (US); Tushar Bhangale, South San Francisco, CA (US)

(21) Appl. No.: 14/357,192

(22) PCT Filed: Nov. 9, 2012
(86) PCT No.: PCT/US2012/064466

§ 371 Date: May 8, 2014

Related U.S. Application Data
(60) Provisional application No. 61/558,197, filed on Nov. 10, 2011.
(60) Provisional application No. 61/671,531, filed on Jul. 13, 2012.

(51) Int. Cl. C12Q 1/68

(52) U.S. Cl. CPC C12Q 1/6827
USPC 506/9; 435/6.11; 506/16; 536/23.5

(57) ABSTRACT

The invention provides methods of diagnosis and prognosis of Alzheimer' disease (AD) in a subject comprising detecting the presence or absence of one or more genetic variations in a sample from the subject, wherein the presence of the genetic variation indicates that the subject is afflicted with, or at risk of developing, AD. Methods of predicting the response of a subject to therapeutic agents for the treatment of AD are also provided.

### FIELD OF THE INVENTION

Methods of identifying, diagnosing, and prognosing Alzheimer's Disease (AD), including certain subphenotypes of AD, are provided, as well as methods of treating AD, including certain subpopulations of patients. Also provided are methods for identifying effective AD therapeutic agents and predicting responsiveness to AD therapeutic agents.

### BACKGROUND

Alzheimer's Disease (AD) is a neurodegenerative disease of the central nervous system associated with progressive loss of cognitive and memory function, and ultimately dementia. AD is the most significant and common cause of dementia in developed countries, accounting for 60% or more of all cases of dementia. Two pathological characteristics are observed in AD patients at autopsy: extracellular plaques and intracellular tangles in the hippocampus, cerebral cortex, and other areas of the brain essential for cognitive function. Plaques are formed mostly from the deposition of amyloid β (Aβ), a peptide derived from amyloid precursor protein (APP).

The frequency of AD increases with each decade of adult life, reaching 20-40% of the population over the age of 85. Because more and more people will live into their 80's and 90's, the number of patients is expected to triple over the next 20 years. More than 5 million people suffer from AD in the USA, where 800,000 deaths per year are associated with AD. In 2011, the cost of caring for AD patients is estimated to be a total of $183 billion dollars. AD also puts a heavy emotional toll on family members and caregivers: about 14.9 million people care for AD patients in the USA. AD patients live for an average of 7 to 10 years after diagnosis and spend an average of 5 years under care either at home or in a nursing home.

Early-onset Alzheimer's disease (EOAD) is a rare form of Alzheimer's disease in which individuals are diagnosed with the disease before age 65. Less than 10% of all Alzheimer's disease patients have EOAD. Approximately half the cases of EOAD are familial, in which disease inheritance follows an autosomal dominant pattern. AD cases in which no obvious inheritance pattern is found are termed “sporadic.” To date, mutations in three genes including amyloid precursor protein (APP) on chromosome 21, presenilin 1 (PSEN1) on chromosome 14 and presenilin 2 (PSEN2) on chromosome 1 have been identified in families with familial EOAD. Most of the pathogenic mutations in the APP and presenilin genes are associated with abnormal processing of APP, which leads to an increase in the production of Aβ42, the main component in amyloid plaques.

Late-onset Alzheimer's disease (LOAD) is the most common form of Alzheimer's disease, accounting for about 90% of cases and usually occurring after age 65. LOAD strikes almost half of all individuals over the age of 85, and is typically sporadic. Based on twin studies, heritability for the disease has been estimated at 79%, with no difference (after controlling for age) between men and women in prevalence or heritability (Gatz, et al., Arch. Gen. Psychiatry, 63:168-74 (2006)). The single-gene mutations identified to date as being associated with early-onset Alzheimer's disease do not seem to be involved in late-onset Alzheimer's.

While no specific gene has been found that causes the late-onset form of AD, one genetic risk factor that increases a person's risk of developing the disease is related to the apolipoprotein E (APOE) gene found on chromosome 19. Early genetic studies of AD demonstrated association and linkage to the same region on chromosome 19 containing the APOE gene (Schellenberg, et al., J. Neurogenet., 4:97-108 (1987); Pericak-Vance, et al., Am. J. Hum. Gen., 48:1034-1050 (1991)). The APOE gene has three common alleles, designated ε2, ε3, and ε4. As compared to the common ε3 allele, the ε4 allele increases the risk of AD, while the ε2 allele decreases the risk of AD. (Corder, et al. (1993) Science, 281:921-923; Corder et al. (1994) Nat. Genet. 7: 180-184). While the lifetime risk (LTR) of AD by age 85 for the general population is 11-14%, the LTR rises to 23-35% for APOE 3/4 carriers, and to 51-68% for APOE 4/4 carriers (Genin et al. (2011) Molecular Psychiatry 16: 903-907). The AD risk for APOE 2/4 carriers is the same as for subjects having the neutral genotype APOE 3/3, while APOE 2/3 carriers have a decreased risk. Although 40-65% of AD patients have at least one copy of the APOE-ε4 allele, APOE-ε4 is not a required determinant of the disease in that at least a third of patients with AD are APOE-ε4 negative and some APOE-ε4 homozygotes never develop the disease. Thus this allele on its own is not sufficient for diagnosis of AD (Ertekin-Taner (2007) Neurol. Clin. 25: 811).

Currently, the primary method of diagnosing AD involves taking detailed patient histories, administering memory and psychological tests, and ruling out other explanations for memory loss, including temporary (e.g., depression or vitamin B12 deficiency) or permanent (e.g., stroke) conditions. Under this approach, AD cannot be conclusively diagnosed until after death, when autopsy reveals the disease's characteristic amyloid plaques and neurofibrillary tangles in a patient's brain. In addition, clinical diagnostic procedures are only helpful after patients have begun displaying significant, abnormal memory loss or personality changes. By then, a patient has likely had AD for years. A diagnostic test that, for example, enables physicians to identify AD early in the disease process, or identify individuals who are at high risk of developing the disease, will provide the option to intervene at an early stage in the disease process. Early intervention in disease processes does generally result in better treatment results by delaying disease onset or progression compared to later intervention. There is therefore a need for other methods of diagnosing and aiding diagnosis of AD.

### SUMMARY

The invention provides methods of diagnosis and prognosis of Alzheimer' disease (AD) in a subject comprising detecting the presence or absence of one or more genetic variations in a sample from the subject, wherein the presence of the genetic variation indicates that the subject is afflicted with, or at risk of developing, AD as disclosed herein.

In an embodiment, the invention provides a method of screening for genetic variants having a detrimental or beneficial effect on the development of AD in subjects having at least one APOE-ε4 allele, the method comprising identifying a genetic variant that is present at increased or decreased frequency in subjects under 65 years of age, having AD, and having at least one APOE-ε4 allele, as compared to control subjects over 75 years of age, without AD, and having at least one APOE-ε4 allele, wherein increased frequency in subjects having AD as compared to control subjects indicates that the genetic variation is associated with a detrimental effect in subjects having at least one APOE-ε4 allele, and decreased frequency in subjects having AD as compared to control subjects indicates that the genetic variation is associated with a beneficial effect in subjects having at least one APOE-ε4 allele. In some embodiments, the genetic variation is identified using a genome-wide association scan.

The invention further provides a method of screening for genetic variants having a detrimental or beneficial effect on the development of AD in subjects having at least one APOE-ε4 allele, the method comprising (a) determining the genotype at one or more genetic locus of a plurality of subjects under 65 years of age, having AD, and having at least one APOE-ε4 allele; (b) determining the genotype at one or more genetic locus of a plurality of control subjects over 75 years of age, without AD, and having at least one APOE-ε4 allele; and (c) identifying a genetic variant that is present at increased or decreased frequency in subjects having AD as compared to control subjects, wherein increased frequency in subjects having AD as compared to control subjects indicates that the genetic variation is associated with a detrimental effect in subjects having at least one APOE-ε4 allele, and decreased frequency in subjects having AD as compared to control subjects indicates that the genetic variation is associated with a beneficial effect in subjects having at least one APOE-ε4 allele.

In some embodiments of these screening methods, the detrimental effect is increased risk of developing AD. In some embodiments, the detrimental effect is lower age of onset of AD. In some embodiments, the beneficial effect is decreased risk of developing AD. In some embodiments, the beneficial effect is later age of onset of AD.

In an embodiment, the invention provides a method for detecting the presence or absence of a genetic variation indicative of Alzheimer's disease (AD) in a subject, comprising: (a) contacting a sample from the subject with a reagent capable of detecting the presence or absence of a genetic variation in a gene selected from the genes encoding IL6R, NTF4 and UNC5C, or a gene product thereof; and (b) determining the presence or absence of the genetic variation, wherein the presence of the genetic variation indicates that the subject is afflicted with, or at risk of developing, AD.

In various embodiments, the at least one genetic variation is a single nucleotide polymorphism (SNP), an allele, a haplotype, an insertion, or a deletion. In some embodiments, the genetic variation is a SNP. In an embodiment, the genetic variation is a SNP that results in the amino acid substitution D358A in the amino acid sequence of IL6R (SEQ ID NO:1). In a further embodiment, the genetic variation is a ‘C’ allele at rs2228145. In an embodiment, the genetic variation is a SNP that results in the amino acid substitution R206W in the amino acid sequence of NTF4 (SEQ ID NO:2). In a further embodiment, the genetic variation is a ‘T’ allele at rs121918427. In an embodiment, the genetic variation is a SNP that results in the amino acid substitution T835M in the amino acid sequence of UNCSC (SEQ ID NO:3). In a further embodiment, the genetic variation is a SNP that substitutes G for A in the codon encoding the amino acid at position 835 of UNCSC (SEQ ID NO:3).

In other embodiments, the at least one genetic variation is an amino acid substitution, insertion, or deletion. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNCSC (SEQ ID NO:3).

In some embodiments of the method, the reagent is selected from an oligonucleotide, a DNA probe, an RNA probe, and a ribozyme. In other embodiments, the reagent is an antibody that specifically binds to a protein comprising the genetic variation. In some embodiments, the reagent is labeled.

In some embodiments of the method, the sample is selected from one of cerebrospinal fluid, blood, serum, sputum, saliva, mucosal scraping, tissue biopsy, lacrimal secretion, semen, or sweat.

In an embodiment, the method further comprises treating the subject for AD based on the results of step (b). In an embodiment, the method further comprises detecting in the sample the presence of at least one APOE-ε4 allele. In an embodiment, the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic variation.

The invention further provides a method for detecting a genetic variation indicative of Alzheimer's disease (AD) in a subject, comprising: determining the presence or absence of a genetic variation in a gene selected from the genes encoding IL6R, NTF4 and UNC5C, or a gene product thereof, in a biological sample from a subject, wherein the presence of the genetic variation indicates that the subject is afflicted with, or at risk of developing, AD.

In various embodiments, the at least one genetic variation is a single nucleotide polymorphism (SNP), an allele, a haplotype, an insertion, or a deletion. In some embodiments, the genetic variation is a SNP. In an embodiment, the genetic variation is a SNP that results in the amino acid substitution D358A in the amino acid sequence of IL6R (SEQ ID NO:1). In a further embodiment, the genetic variation is a ‘C’ allele at rs2228145. In an embodiment, the genetic variation is a SNP that results in the amino acid substitution R206W in the amino acid sequence of NTF4 (SEQ ID NO:2). In a further embodiment, the genetic variation is a ‘T’ allele at rs121918427. In an embodiment, the genetic variation is a SNP that results in the amino acid substitution T835M in the amino acid sequence of UNC5C (SEQ ID NO:3). In a further embodiment, the genetic variation is a SNP that substitutes G for A in the codon encoding for the amino acid at position 835 of UNC5C (SEQ ID NO:3).

In other embodiments, the at least one genetic variation is an amino acid substitution, insertion, or deletion. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNC5C (SEQ ID NO:3).

In various embodiments of the method, detection of the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism. In some embodiments, nucleic acids from the sample are amplified prior to determining the presence of the one or more genetic variation.

In other embodiments of the method, detection of the presence of the one or more genetic variation in a protein is carried out by a process selected from electrophoresis, chromatography, mass spectroscopy, proteolytic digestion, protein sequencing, immunoaffinity assay, or a combination thereof. In some embodiments, proteins from the sample are purified using antibodies or peptides that bind the proteins prior to determining the presence of the one or more genetic variation.

In some embodiments of the method, the sample is selected from one of cerebrospinal fluid, blood, serum, sputum, saliva, mucosal scraping, tissue biopsy, lacrimal secretion, semen, or sweat.

In an embodiment, the method further comprises treating the subject for AD based on the results of step (b). In an embodiment, the method further comprises detecting in the sample the presence of at least one APOE-ε4 allele. In an embodiment, the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

The invention further provides a method for diagnosing or prognosing AD in a subject, comprising: (a) contacting a sample from the subject with a reagent capable of detecting the presence or absence of a genetic variation in a gene selected from the genes encoding IL6R, NTF4 and UNC5C, or a gene product thereof; and (b) determining the presence or absence of the genetic variation, wherein the presence of the genetic variation indicates that the subject is afflicted with, or at risk of developing, AD.

In various embodiments, the at least one genetic variation is a single nucleotide polymorphism (SNP), an allele, a haplotype, an insertion, or a deletion. In some embodiments, the genetic variation is a SNP. In an embodiment, the genetic variation is a SNP that results in the amino acid substitution D358A in the amino acid sequence of IL6R (SEQ ID NO:1). In a further embodiment, the genetic variation is a ‘C’ allele at rs2228145. In an embodiment, the genetic variation is a SNP that results in the amino acid substitution R206W in the amino acid sequence of NTF4 (SEQ ID NO:2). In a further embodiment, the genetic variation is a ‘T’ allele at rs121918427. In an embodiment, the genetic variation is a SNP that results in the amino acid substitution T835M in the amino acid sequence of UNC5C (SEQ ID NO:3). In a further embodiment, the genetic variation is a SNP that substitutes G for A in the codon encoding for the amino acid at position 835 of UNC5C (SEQ ID NO:3).

In other embodiments, the at least one genetic variation is an amino acid substitution, insertion, or deletion. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNC5C (SEQ ID NO:3).

In some embodiments of the method, the reagent is selected from an oligonucleotide, a DNA probe, an RNA probe, and a ribozyme. In other embodiments, the reagent is an antibody that specifically binds to a protein comprising the genetic variation. In some embodiments, the reagent is labeled.

In some embodiments of the method, the sample is selected from one of cerebrospinal fluid, blood, serum, sputum, saliva, mucosal scraping, tissue biopsy, lacrimal secretion, semen, or sweat.

In an embodiment, the method further comprises treating the subject for AD based on the results of step (b). In an embodiment, the method further comprises detecting in the sample the presence of at least one APOE-ε4 allele. In an embodiment, the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

In some embodiments, the method further comprises subjecting the subject to one or more additional diagnostic tests for AD selected from the group consisting of screening for one or more additional genetic markers, administering a mental status exam, or subjecting the subject to imaging procedures.

In some embodiments, the method further comprises analyzing the sample to detect the presence of at least one additional genetic marker that is an APOE modifier, wherein the at least one additional genetic marker is in a gene selected from the gene encoding IL6R, the gene encoding NTF4, the gene encoding UNC5C, and a gene listed in Table 3. In various embodiments, the at least one additional genetic marker is a SNP that results in the amino acid substitution D358A in the amino acid sequence of IL6R (SEQ ID NO:1), a SNP that results in the amino acid substitution R206W in the amino acid sequence of NTF4 (SEQ ID NO:2), a SNP that results in the amino acid substitution T835W in the amino acid sequence of UNC5C (SEQ ID NO:3), or a SNP that is listed in Table 3.

The invention further provides a method of diagnosing or prognosing AD in a subject, comprising: determining the presence or absence of a genetic variation in a gene selected from the genes encoding IL6R, NTF4 and UNC5C, or a gene product thereof, in a biological sample from a subject, wherein the presence of the genetic variation indicates that the subject is afflicted with, or at risk of developing, AD.

In various embodiments, the at least one genetic variation is a single nucleotide polymorphism (SNP), an allele, a haplotype, an insertion, or a deletion. In some embodiments, the genetic variation is a SNP. In an embodiment, the genetic variation is a SNP that results in the amino acid substitution D358A in the amino acid sequence of IL6R (SEQ ID NO:1). In a further embodiment, the genetic variation is a ‘C’ allele at rs2228145. In an embodiment, the genetic variation is a SNP that results in the amino acid substitution R206W in the amino acid sequence of NTF4 (SEQ ID NO:2). In a further embodiment, the genetic variation is a allele at rs121918427. In an embodiment, the genetic variation is a SNP that results in the amino acid substitution T835M in the amino acid sequence of UNC5C (SEQ ID NO:3). In a further embodiment, the genetic variation is a SNP that substitutes G for A in the codon encoding for the amino acid at position 835 of UNC5C (SEQ ID NO:3).

In other embodiments, the at least one genetic variation is an amino acid substitution, insertion, or deletion. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNC5C (SEQ ID NO:3).

In various embodiments of the method, detection of the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism. In some embodiments, nucleic acids from the sample are amplified prior to determining the presence of the one or more genetic variation.

In other embodiments of the method, detection of the presence of the one or more genetic variation in a protein is carried out by a process selected from electrophoresis, chromatography, mass spectroscopy, proteolytic digestion, protein sequencing, immunoaffinity assay, or a combination thereof. In some embodiments, proteins from the sample are purified using antibodies or peptides that bind the proteins prior to determining the presence of the one or more genetic variation.

In some embodiments of the method, the sample is selected from one of cerebrospinal fluid, blood, serum, sputum, saliva, mucosal scraping, tissue biopsy, lacrimal secretion, semen, or sweat.

In an embodiment, the method further comprises treating the subject for AD based on the results of step (b). In an embodiment, the method further comprises detecting in the sample the presence of at least one APOE-ε4 allele. In an embodiment, the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

In some embodiments, the method further comprises analyzing the sample to detect the presence of at least one additional genetic marker that is an APOE modifier, wherein the at least one additional genetic marker is in a gene selected from the gene encoding IL6R, the gene encoding NTF4, the gene encoding UNC5C, and a gene listed in Table 3. In various embodiments, the at least one additional genetic marker is a SNP that results in the amino acid substitution D358A in the amino acid sequence of IL6R (SEQ ID NO:1), a SNP that results in the amino acid substitution R206W in the amino acid sequence of NTF4 (SEQ ID NO:2), a SNP that results in the amino acid substitution T835W in the amino acid sequence of UNC5C (SEQ ID NO:3), or a SNP that is listed in Table 3.

The invention further provides a method of identifying a subject having an increased risk of earlier age of onset of AD, comprising: (a) determining the presence or absence of a genetic variation in a gene selected from the genes encoding IL6R, NTF4 and UNC5C, or a gene product thereof, in a biological sample from a subject; and (b) determining the presence or absence of at least one APOE-ε4 allele, wherein the presence of the genetic variation and at least one APOE-ε4 allele indicates that the subject has an increased risk of earlier age of diagnosis of AD as compared to a subject lacking the presence of the genetic variation and at least one APOE-ε4 allele.

In various embodiments, the at least one genetic variation is a single nucleotide polymorphism (SNP), an allele, a haplotype, an insertion, or a deletion. In some embodiments, the genetic variation is a SNP. In an embodiment, the genetic variation is a SNP that results in the amino acid substitution D358A in the amino acid sequence of IL6R (SEQ ID NO:1). In a further embodiment, the genetic variation is a ‘C’ allele at rs2228145. In an embodiment, the genetic variation is a SNP that results in the amino acid substitution R206W in the amino acid sequence of NTF4 (SEQ ID NO:2). In a further embodiment, the genetic variation is a ‘T’ allele at rs121918427. In an embodiment, the genetic variation is a SNP that results in the amino acid substitution T835M in the amino acid sequence of UNC5C (SEQ ID NO:3). In a further embodiment, the genetic variation is a SNP that substitutes G for A in the codon encoding for the amino acid at position 835 of UNC5C (SEQ ID NO:3).

In other embodiments, the at least one genetic variation is an amino acid substitution, insertion, or deletion. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNC5C (SEQ ID NO:3).

In various embodiments of the method, detection of the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism. In some embodiments, nucleic acids from the sample are amplified prior to determining the presence of the one or more genetic variation.

In other embodiments of the method, detection of the presence of the one or more genetic variation in a protein is carried out by a process selected from electrophoresis, chromatography, mass spectroscopy, proteolytic digestion, protein sequencing, immunoaffinity assay, or a combination thereof. In some embodiments, proteins from the sample are purified using antibodies or peptides that bind the proteins prior to determining the presence of the one or more genetic variation.

In some embodiments of the method, the sample is selected from one of cerebrospinal fluid, blood, serum, sputum, saliva, mucosal scraping, tissue biopsy, lacrimal secretion, semen, or sweat.

The invention further provides a method of aiding prognosis of a subphenotype of AD in a subject, the method comprising detecting in a biological sample derived from the subject the presence of a SNP that results in the amino acid substitution D358A in the amino acid sequence of IL6R (SEQ ID NO:1), wherein the subphenotype of AD is characterized at least in part by increased levels of soluble IL6R in a biological sample derived from the subject as compared to one or more control subjects.

The invention further provides a method of predicting the response of a subject to an AD therapeutic agent that targets IL6R, comprising detecting in a biological sample obtained from the subject a SNP that results in the amino acid substitution D358A in the amino acid sequence of IL6R (SEQ ID NO:1), wherein the presence of the SNP is indicative of a response to a therapeutic agent that targets IL6R. In an embodiment, the therapeutic agent is an anti-IL6R antibody.

The invention further provides a method of aiding prognosis of a subphenotype of AD in a subject, the method comprising detecting in a biological sample derived from the subject the presence of a SNP that results in the amino acid substitution R206W in the amino acid sequence of NTF4 (SEQ ID NO:2), wherein the subphenotype of AD is characterized at least in part by decreased activation of TrkB in a biological sample derived from the subject as compared to one or more control subjects.

The invention further provides a method of predicting the response of a subject to an AD therapeutic agent that targets TrkB, comprising detecting in a biological sample obtained from the subject a SNP that results in the amino acid substitution R206W in the amino acid sequence of NTF4 (SEQ ID NO:2), wherein the presence of the SNP is indicative of a response to a therapeutic agent that targets TrkB. In an embodiment, the therapeutic agent is a TrkB agonist.

The invention further provides a method of aiding prognosis of a subphenotype of AD in a subject, the method comprising detecting in a biological sample derived from the subject the presence of a SNP that results in the amino acid substitution T835M in the amino acid sequence of UNC5C (SEQ ID NO:3), wherein the subphenotype of AD is characterized at least in part by increased apoptotic activity of UNC5C in a biological sample derived from the subject as compared to one or more control subjects.

The invention further provides a method of predicting the response of a subject to an AD therapeutic agent that targets UNC5C, comprising detecting in a biological sample obtained from the subject a SNP that results in the amino acid substitution T835M in the amino acid sequence of UNC5C (SEQ ID NO:3), wherein the presence of the SNP is indicative of a response to a therapeutic agent that targets UNC5C. In an embodiment, the therapeutic agent targets the UNC5C death domain.

The invention further provides a method of diagnosing or prognosing Alzheimer's Disease (AD) in a subject, comprising: (a) contacting a sample from the subject with a reagent capable of detecting the presence or absence of one or more SNPs selected from the group consisting of a SNP that results in the amino acid substitution D358A in the amino acid sequence of IL6R (SEQ ID NO:1), a SNP that results in the amino acid substitution R206W in the amino acid sequence of NTF4, and a SNP that results in the amino acid substitution T835M in the amino acid sequence of UNC5C (SEQ ID NO:3), and (b) analyzing the sample to detect the presence of said one or more SNPs, wherein the presence of the one or more SNPs in the sample indicates that the subject is afflicted with, or at risk of developing, AD. In an embodiment, the method further comprises detecting one or more SNPs selected from the SNPs listed in Table 3.

The invention further provides a kit for carrying out the method, comprising at least one oligonucleotide detection reagent, wherein the oligonucleotide detection reagent distinguishes between each of at least two different alleles at the one or more SNP. In various embodiments, the detecting is carried out by a process selected from the group consisting of direct sequencing, allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

In an embodiment, the oligonucleotide detection reagents are immobilized to a substrate. In a further embodiment, the oligonucleotide detection reagents are arranged on an array.

The invention further provides a method of diagnosing or prognosing Alzheimer's Disease (AD) in a subject, comprising: (a) contacting a sample from the subject with a reagent capable of detecting the presence or absence of one or more amino acid substitutions selected from the group consisting of the amino acid substitution D358A in the amino acid sequence of IL6R (SEQ ID NO:1), the amino acid substitution R206W in the amino acid sequence of NTF4, and the amino acid substitution T835M in the amino acid sequence of UNC5C (SEQ ID NO:3), and (b) analyzing the sample to detect the presence of said one or more amino acid substitutions, wherein the presence of the one or more amino acid substitutions in the sample indicates that the subject is afflicted with, or at risk of developing, AD.

The invention further provides a kit for carrying out the method, comprising at least one antibody detection reagent, wherein the antibody detection reagent distinguishes between each of at least two different amino acids at the one or more amino acid substitution. The invention further provides a kit for carrying out the method, comprising at least one peptide detection reagent, wherein the peptide detection reagent distinguishes between each of at least two different amino acids at the one or more amino acid substitution.

The invention further provides a therapeutic target for the treatment of AD, wherein the therapeutic target is one or a combination of proteins encoded by the genes selected from IL6R, NTF4 and UNC5C.

The invention further provides a set of molecular probes for diagnosis or prognosing AD comprising at least two probes capable of detecting directly or indirectly at least two markers selected from the group comprising: a SNP that results in the amino acid substitution D358A in the amino acid sequence of IL6R (SEQ ID NO:1), a SNP that results in the amino acid substitution R206W in the amino acid sequence of NTF4, and a SNP that results in the amino acid substitution T835M in the amino acid sequence of UNC5C (SEQ ID NO:3), wherein said molecular probes are not associated with a microarray of greater than 1000 elements. In an embodiment, the set of molecular probes further comprises one or more probes capable of detecting directly or indirectly at least two markers selected from the SNPs listed in Table 3.

### BRIEF DESCRIPTION OF THE FIGURES

FIG. 1 illustrates the strategy used in the APOE modifier screen.

FIG. 2 is a Manhattan plot showing the locations across a portion of human chromosome 1 where there was a statistically significant difference between genetic variants in the AD case samples versus the supercontrols. The lower the p-value, the stronger the association.

FIG. 3 shows the frequency of the T allele of rs4129267, proxy of the C allele of rs2228145, in unselected Alzheimer's disease cases (N=932 individuals) and controls (N=832 individuals) from the NIA/LOAD study. The frequency of the minor allele is stratified by age of onset in AD cases and age in controls.

FIG. 4 shows an analysis of data from the TGEN project (Webster et al. (2009) Am. J. Hum. Genet. 84: 445-458). Expression levels of both membrane bound and soluble IL6R in the brains of subjects with AD (AD) were compared to controls (CN), using either a probe that detects only the membrane bound form of IL6R (NM_{—}000565), or a probe that captures both the membrane bound and sIL6R(NM_{—}181359).

FIG. 5 shows the results of nonparametric linkage analysis in the LO1 pedigree

FIG. 6 provides an amino acid sequence alignment of UNC5 family members, showing the conservation of amino acid residue T853.

FIG. 7 is a Manhattan plot showing the locations across a portion of human chromosome 1 where there was a statistically significant association between genetic variants and levels of soluble IL6R in cerebrospinal fluid. The lower the p-value, the stronger the association.

FIG. 8 shows the relative membrane bound percentage of IL6R in 293T cells transfected with D358 or A358 constructs of IL6R, and treated with 100 nM phorbol myristate acetate (PMA) for 0, 30, 60 or 120 minutes. Cells were harvested after treatment, stained with an IL6R-PE antibody, and membrane bound IL6R was analyzed by FACS.

FIG. 9 shows the percentage of membrane-bound IL6R in CD4⁺ T cells from age, gender and ethnicity matched donors that were homozygous for either D358 or A358, before and after treatment with 100 nM PMA for 60 min. Cells were harvested soon after treatment, stained with an IL6R-PE antibody, and membrane bound IL6R was analyzed by FACS.

FIG. 10 shows soluble IL6R for human CD4⁺ T cells from age, gender and ethnicity matched donors that were homozygous for either D358 or A358. The CD4⁺ T cells were plated on anti-CD3/anti-CD28, harvested after 24, 48 and 72 hours for total RNA extraction, and the supernatant collected to determine the sIL6R levels by ELISA. The graph shows the fold increase in soluble IL6R for A358, relative to D358, at each time point.

### DETAILED DESCRIPTION OF EMBODIMENTS OF THE INVENTION

### Definitions

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′-OH group.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “genetic variation” or “nucleotide variation” refers to a change in a nucleotide sequence (e.g., an insertion, deletion, inversion, or substitution of one or more nucleotides, such as a single nucleotide polymorphism (SNP)) relative to a reference sequence (e.g., a commonly-found and/or wild-type sequence, and/or the sequence of a major allele). The term also encompasses the corresponding change in the complement of the nucleotide sequence, unless otherwise indicated. In one embodiment, a genetic variation is a somatic polymorphism. In one embodiment, a genetic variation is a germline polymorphism.

A “single nucleotide polymorphism”, or “SNP”, refers to a single base position in DNA at which different alleles, or alternative nucleotides, exist in a population. The SNP position is usually preceded by and followed by highly conserved sequences of the allele (e.g., sequences that vary in less than 1/100 or 1/1000 members of the populations). An individual may be homozygous or heterozygous for an allele at each SNP position.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “a genetic variation at a nucleotide position corresponding to a SNP,” “a nucleotide variation at a nucleotide position corresponding to a SNP,” and grammatical variants thereof refer to a nucleotide variation in a polynucleotide sequence at the relative corresponding DNA position occupied by said SNP in the genome. The term also encompasses the corresponding variation in the complement of the nucleotide sequence, unless otherwise indicated.

As used herein, the term “allele” refers to one of a pair or series, of forms of a gene or non-genic region that occur at a given locus in a chromosome. In a normal diploid cell there are two alleles of any one gene (one from each parent), which occupy the same relative position (locus) on homologous chromosomes. Within a population there may be more than two alleles of a gene. SNPs also have alleles, i.e., the two (or more) nucleotides that characterize the SNP.

As used herein, the term “linkage disequilibrium” or “LD” refers to the situation in which the alleles for two or more loci do not occur together in individuals sampled from a population at frequencies predicted by the product of their individual allele frequencies. Markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait.

As used herein, the term “locus” refers to a specific position along a chromosome or DNA sequence. Depending upon context, a locus could be a gene, a marker, a chromosomal band or a specific sequence of one or more nucleotides.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., the polymerase chain reaction (PCR)). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “allele-specific oligonucleotide” refers to an oligonucleotide that hybridizes to a region of a target nucleic acid that comprises a nucleotide variation (often a substitution). “Allele-specific hybridization” means that, when an allele-specific oligonucleotide is hybridized to its target nucleic acid, a nucleotide in the allele-specific oligonucleotide specifically base pairs with the nucleotide variation. An allele-specific oligonucleotide capable of allele-specific hybridization with respect to a particular nucleotide variation is said to be “specific for” that variation.

The term “allele-specific primer” refers to an allele-specific oligonucleotide that is a primer.

The term “primer extension assay” refers to an assay in which nucleotides are added to a nucleic acid, resulting in a longer nucleic acid, or “extension product,” that is detected directly or indirectly. The nucleotides can be added to extend the 5′ or 3′ end of the nucleic acid.

The term “allele-specific nucleotide incorporation assay” refers to a primer extension assay in which a primer is (a) hybridized to target nucleic acid at a region that is 3′ or 5′ of a nucleotide variation and (b) extended by a polymerase, thereby incorporating into the extension product a nucleotide that is complementary to the nucleotide variation.

The term “allele-specific primer extension assay” refers to a primer extension assay in which an allele-specific primer is hybridized to a target nucleic acid and extended.

The term “allele-specific oligonucleotide hybridization assay” refers to an assay in which (a) an allele-specific oligonucleotide is hybridized to a target nucleic acid and (b) hybridization is detected directly or indirectly.

The term “5′ nuclease assay” refers to an assay in which hybridization of an allele-specific oligonucleotide to a target nucleic acid allows for nucleolytic cleavage of the hybridized probe, resulting in a detectable signal.

The term “assay employing molecular beacons” refers to an assay in which hybridization of an allele-specific oligonucleotide to a target nucleic acid results in a level of detectable signal that is higher than the level of detectable signal emitted by the free oligonucleotide.

The term “oligonucleotide ligation assay” refers to an assay in which an allele-specific oligonucleotide and a second oligonucleotide are hybridized adjacent to one another on a target nucleic acid and ligated together (either directly or indirectly through intervening nucleotides), and the ligation product is detected directly or indirectly.

The term “target sequence,” “target nucleic acid,” or “target nucleic acid sequence” refers generally to a polynucleotide sequence of interest in which a nucleotide variation is suspected or known to reside, including copies of such target nucleic acid generated by amplification.

The term “detection” includes any means of detecting, including direct and indirect detection.

The term “IL6R” is used to refer to the interleukin-6 receptor, which is also known as IL-6R1, IL-6RA, IL-6R alpha, interleukin-6 receptor subunit alpha, and CD126. The term encompasses “full-length,” unprocessed IL6R as well as any form of IL6R that results from processing in the cell. The term also encompasses naturally occurring variants of IL6R, e.g., splice variants or allelic variants. The amino acid sequence of an exemplary human IL6R is shown in SEQ ID NO:1:

(SEQ ID NO: 1)
MLAVGCALLAALLAAPGAALAPRRCPAQEVARGVLTSLPGDSVTLTC
PGVEPEDNATVHWVLRKPAAGSHPSRWAGMGRRLLLRSVQLHDSGNY
SCYRAGRPAGTVHLLVDVPPEEPQLSCFRKSPLSNVVCEWGPRSTPS
LTTKAVLLVRKFQNSPAEDFQEPCQYSQESQKFSCQLAVPEGDSSFY
IVSMCVASSVGSKFSKTQTFQGCGILQPDPPANITVTAVARNPRWLS
VTWQDPHSWNSSFYRLRFELRYRAERSKTFTTWMVKDLQHHCVIHDA
WSGLRHVVQLRAQEEFGQGEWSEWSPEAMGTPWTESRSPPAENEVST
PMQALTTNKDDDNILFRDSANATSLPVQDSSSVPLPTFLVAGGSLAF
GTLLCIAIVLRFKKTWKLRALKEGKTSMHPPYSLGQLVPERPRPTPV
LVPLISPPVSPSSLGSDNTSSHNRPDARDPRSPYDISNTDYFFPR
(Genbank Accession No. NP_000566).

The term “NTF4” is used to refer to neutrotrophin 4, which is also known as neutrotrophin 5, neurotrophic factor 4, neurotrophic factor 5, NT4, NT5, NT-4, NT-5, NTF5, GLC10 and NT-4/5. The term encompasses “full-length,” unprocessed NTF4 as well as any form of NTF4 that results from processing in the cell. The term also encompasses naturally occurring variants of NTF4, e.g., splice variants or allelic variants. The amino acid sequence of an exemplary human NTF4 is shown in SEQ ID NO:2:

(SEQ ID NO: 2)
MLPLPSCSLPILLLFLLPSVPIESQPPPSTLPPFLAPEWDLLSPRVV
LSRGAPAGPPLLFLLEAGAFRESAGAPANRSRRGVSETAPASRRGEL
AVCDAVSGWVTDRRTAVDLRGREVEVLGEVPAAGGSPLRQYFFETRC
KADNAEEGGPGAGGGGCRGVDRRHWVSECKAKQSYVRALTADAQGRV
GWRWIRIDTACVCTLLSRTGRA (Genbank Accession No.
NP_006170).

The term “UNC5C” is used to refer to netrin receptor UNC5C, which is also known as UNC-5 homolog 3, UNC-5 homolog C, and UNC5H3. The term encompasses “full-length,” unprocessed UNC5C as well as any form of UNC5C that results from processing in the cell. The term also encompasses naturally occurring variants of UNC5C, e.g., splice variants or allelic variants. The amino acid sequence of an exemplary human UNC5C is shown in SEQ ID NO:3:

(SEQ ID NO: 3)
MRKGLRATAARCGLGLGYLLQMLVLPALALLSASGTGSAAQDDDFFH
ELPETFPSDPPEPLPHFLIEPEEAYIVKNKPVNLYCKASPATQIYFK
CNSEWVHQKDHIVDERVDETSGLIVREVSIEISRQQVEELFGPEDYW
CQCVAWSSAGTTKSRKAYVRIAYLRKTFEQEPLGKEVSLEQEVLLQC
RPPEGIPVAEVEWLKNEDIIDPVEDRNFYITIDHNLIIKQARLSDTA
NYTCVAKNIVAKRKSTTATVIVYVNGGWSTWTEWSVCNSRCGRGYQK
RTRTCTNPAPLNGGAFCEGQSVQKIACTTLCPVDGRWTPWSKWSTCG
TECTHWRRRECTAPAPKNGGKDCDGLVLQSKNCTDGLCMQTAPDSDD
VALYVGIVIAVIVCLAISVVVALFVYRKNHRDFESDIIDSSALNGGF
QPVNIKAARQDLLAVPPDLTSAAAMYRGPVYALHDVSDKIPMTNSPI
LDPLPNLKIKVYNTSGAVTPQDDLSEFTSKLSPQMTQSLLENEALSL
KNQSLARQTDPSCTAFGSFNSLGGHLIVPNSGVSLLIPAGAIPQGRV
YEMYVTVHRKETMRPPMDDSQTLLTPVVSCGPPGALLTRPVVLTMHH
CADPNTEDWKILLKNQAAQGQWEDVVVVGEENFTTPCYIQLDAEACH
ILTENLSTYALVGHSTTKAAAKRLKLAIFGPLCCSSLEYSIRVYCLD
DTQDALKEILHLERQMGGQLLEEPKALHFKGSTHNLRLSIHDIAHSL
WKSKLLAKYQEIPFYHVWSGSQRNLHCTFTLERFSLNTVELVCKLCV
RQVEGEGQIFQLNCTVSEEPTGIDLPLLDPANTITTVTGPSAFSIPL
PIRQKLCSSLDAPQTRGHDWRMLAHKLNLDRYLNYFATKSSPTGVIL
DLWEAQNFPDGNLSMLAAVLEEMGRHETVVSLAAEGQY (Genbank
Accession No. NP_003719).

As used herein the term “Alzheimer's disease” (AD) refers to both early-onset AD and late-onset AD, as well as both familial and sporadic forms of AD.

As used herein, a subject “at risk” of developing Alzheimer's disease may or may not have detectable disease or symptoms of disease, and may or may not have displayed detectable disease or symptoms of disease prior to the treatment methods described herein. “At risk” denotes that a subject has one or more risk factors, which are measurable parameters that correlate with development of Alzheimer's disease, as described herein and known in the art. A subject having one or more of these risk factors has a higher probability of developing Alzheimer's disease than a subject without one or more of these risk factor(s).

The term “diagnosis” is used herein to refer to the identification or classification of a molecular or pathological state, disease or condition, for example, AD. “Diagnosis” may also refer to the classification of a particular sub-type of AD, e.g., by molecular features (e.g., a patient subpopulation characterized by genetic variation(s) in a particular gene or nucleic acid region.)

The term “aiding diagnosis” is used herein to refer to methods that assist in making a clinical determination regarding the presence, or nature, of a particular type of symptom or condition of AD. For example, a method of aiding diagnosis of AD can comprise measuring the presence of absence of one or more genetic markers indicative of AD or an increased risk of having AD in a biological sample from an individual.

The term “prognosis” is used herein to refer to the prediction of the likelihood of developing symptoms, including, for example, memory loss and dementia, of AD. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

An “AD therapeutic agent”, a “therapeutic agent effective to treat AD”, and grammatical variations thereof, as used herein, refer to an agent that when provided in an effective amount is known, clinically shown, or expected by clinicians to provide a therapeutic benefit in a subject who has AD. In one embodiment, the phrase includes any agent that is marketed by a manufacturer, or otherwise used by licensed clinicians, as a clinically-accepted agent that when provided in an effective amount would be expected to provide a therapeutic effect in a subject who has AD. In various non-limiting embodiments, an AD therapeutic agent comprises a cholinesterase inhibitor, memantine, an anti-agitation medication, an anti-depressive, an anxiolytic, or a compound targeting amyloid precursor protein, amyloid beta, amyloid plaques, or any of the enzymes that cleave amyloid precursor protein including, but not limited to alpha-secretase, beta-secretase, and gamma-secretase.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “therapeutic effect,” refers to the production of a condition that is better than the average or normal condition in an individual that is not suffering from a disorder (i.e., a supranormal effect such as improved cognition, memory, mood or other characteristic in a subject attributable at least in part to the functioning of the CNS, as compared to the normal or average state in an unafflicted or asymptomatic subject).

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

A “patient subpopulation,” and grammatical variations thereof, as used herein, refers to a patient subset characterized as having one or more distinctive measurable and/or identifiable characteristics that distinguishes the patient subset from others in the broader disease category to which it belongs. Such characteristics include disease subcategories, gender, lifestyle, health history, organs/tissues involved, treatment history, etc. In one embodiment, a patient subpopulation is characterized by genetic signatures, including genetic variations in particular nucleotide positions and/or regions (such as SNPs).

A “control subject” refers to a healthy subject who has not been diagnosed as having AD and who does not suffer from any sign or symptom associated with AD.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method or composition of the invention is being used to identify. In one embodiment, a reference sample, reference cell, reference tissue, control sample, control cell, or control tissue is obtained from a healthy part of the body of the same subject or patient in whom a disease or condition is being identified using a composition or method of the invention. In one embodiment, a reference sample, reference cell, reference tissue, control sample, control cell, or control tissue is obtained from a healthy part of the body of an individual who is not the subject or patient in whom a disease or condition is being identified using a composition or method of the invention.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured. “Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

A “small molecule” or “small organic molecule” is defined herein as an organic molecule having a molecular weight below about 500 Daltons.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At-211, Cu-67, Bi-212, and Pd-109.

Reference to “about” a value or parameter herein includes (and describes) embodiments that are directed to that value or parameter per se. For example, description referring to “about X” includes description of “X.”

The term “package insert” is used to refer to instructions customarily included in commercial packages of therapeutic products, that contain information about the indications, usage, dosage, administration, combination therapy, contraindications and/or warnings concerning the use of such therapeutic products.

### Compositions and Methods of the Invention

### Genetic Variations

In one aspect, the invention provides methods of detecting the presence or absence of genetic variations associated with Alzheimer's disease (AD) in a sample from a subject, as well as methods of diagnosing and prognosing AD by detecting the presence or absence of one or more of these genetic variations in a sample from a subject, wherein the presence of the genetic variation indicates that the subject is afflicted with, or at risk of developing, AD. Genetic variations associated with AD risk were identified using strategies including genome-wide association studies, modifier screens, and family-based screening.

Genetic variations for use in the methods of the invention include genetic variations in interleukin-6 receptor (IL6R), neurotrophic factor 4 (NTF4) and UNC5C, or the genes encoding these proteins, as well as any of the genes listed in Table 3 or the proteins they encode. In some embodiments, the genetic variation is in genomic DNA that encodes a gene (or its regulatory region) wherein the gene is selected from the genes coding for interleukin-6 receptor (IL6R), neurotrophic factor 4 (NTF4) and UNCSC, and any of the genes listed in Table 3. In various embodiments, the genetic variation is a SNP, an allele, a haplotype, an insertion, or a deletion in one or more genes selected from the genes coding for IL6R, NTF4 and UNCSC, and any of the genes listed in Table 3. In an embodiment, the genetic variation is a SNP that results in the amino acid substitution D358A in the amino acid sequence of IL6R (SEQ ID NO:1). In an embodiment, the genetic variation is a ‘C’ allele at rs2228145. In an embodiment, the genetic variation is a SNP that results in the amino acid substitution R206W in the amino acid sequence of NTF4 (SEQ ID NO:2). In an embodiment, the genetic variation is a ‘T’ allele at rs121918427. In an embodiment, the genetic variation is a SNP that results in the amino acid substitution T835M in the amino acid sequence of UNC5C (SEQ ID NO:3). In embodiments, the genetic variation is a SNP in a gene selected from those listed in Table 3. In embodiments the genetic variation is a SNP selected from rs12733578, rs4658945, rs1478161, rs1024591, rs7799010, rs10969475, and rs12961250. In various embodiments, the at least one genetic variation is an amino acid substitution, insertion, or deletion in IL6R, NTF4 or UNC5C. In some embodiments, the genetic variation is an amino acid substitution. In an embodiment, the genetic variation is the amino acid substitution D358A in the amino acid sequence of IL6R (SEQ ID NO:1). In an embodiment, the genetic variation is the amino acid substitution R206W in the amino acid sequence of NTF4 (SEQ ID NO:2). In an embodiment, the genetic variation is the amino acid substitution T835M in the amino acid sequence of UNC5C (SEQ ID NO:3).

### Detection of Genetic Variations

Nucleic acid, as used in any of the detection methods described herein, may be genomic DNA; RNA transcribed from genomic DNA; or cDNA generated from RNA. Nucleic acid may be derived from a vertebrate, e.g., a mammal. A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine whether a variation is present in the amplicon.

Genetic variations may be detected by certain methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′nuclease assays (e.g., TaqMan™); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., the polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. See, e.g., Wu et al., Genomics 4:560-569 (1989). In addition, a technique known as allele-specific PCR can also be used to detect variations (e.g., substitutions). See, e.g., Ruano and Kidd (1989) Nucleic Acids Research 17:8392; McClay et al. (2002) Analytical Biochem. 301:200-206. In certain embodiments of this technique, an allele-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) allele-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) allele-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′ nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl. Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. See, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. See, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. See, e.g. Orita et al., Proc. Natl. Acad. Sci. USA 86:2766-2770, 1989, and Genomics, 5:874-879, 1989. SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. Denaturing gradient gel electrophoresis (DGGE) differentiates SNP alleles based on the different sequence-dependent stabilities and melting properties inherent in polymorphic DNA and the corresponding differences in electrophoretic migration patterns in a denaturing gradient gel.

Genetic variations may also be detected with the use of microarrays. A microarray is a multiplex technology that typically uses an arrayed series of thousands of nucleic acid probes to hybridize with, e.g, a cDNA or cRNA sample under high-stringency conditions. Probe-target hybridization is typically detected and quantified by detection of fluorophore-, silver-, or chemiluminescence-labeled targets to determine relative abundance of nucleic acid sequences in the target. In typical microarrays, the probes are attached to a solid surface by a covalent bond to a chemical matrix (via epoxy-silane, amino-silane, lysine, polyacrylamide or others). The solid surface is for example, glass, a silicon chip, or microscopic beads. Various microarrays are commercially available, including those manufactured, for example, by Affymetrix, Inc. and Illumina, Inc.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is useful for extremely precise determinations of molecular mass, such as SNPs. Numerous approaches to SNP analysis have been developed based on mass spectrometry. Exemplary mass spectrometry-based methods of SNP genotyping include primer extension assays, which can also be utilized in combination with other approaches, such as traditional gel-based formats and microarrays.

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

In other embodiments of the invention, protein-based detection techniques are used to detect variant proteins encoded by the genes having genetic variations as disclosed herein. Determination of the presence of the variant form of the protein can be carried out using any suitable technique known in the art, for example, electrophoresis (e.g, denaturing or non-denaturing polyacrylamide gel electrophoresis, 2-dimensional gel electrophoresis, capillary electrophoresis, and isoelectrofocusing), chromatrography (e.g., sizing chromatography, high performance liquid chromatography (HPLC), and cation-exchange HPLC), and mass spectroscopy (e.g., MALDI-TOF mass spectroscopy, electrospray ionization (ESI) mass spectroscopy, and tandem mass spectroscopy). See, e.g., Ahrer and Jungabauer (2006) J. Chromatog. B. Analyt. Technol. Biomed. Life Sci. 841: 110-122; and Wada (2002) J. Chromatog. B. 781: 291-301). Suitable techniques may be chosen based in part upon the nature of the variation to be detected. For example, variations resulting in amino acid substitutions where the substituted amino acid has a different charge than the original amino acid, can be detected by isoelectric focusing. Isoelectric focusing of the polypeptide through a gel having a pH gradient at high voltages separates proteins by their pI. The pH gradient gel can be compared to a simultaneously run gel containing the wild-type protein. In cases where the variation results in the generation of a new proteolytic cleavage site, or the abolition of an existing one, the sample may be subjected to proteolytic digestion followed by peptide mapping using an appropriate electrophoretic, chromatographic or, or mass spectroscopy technique. The presence of a variation may also be detected using protein sequencing techniques such as Edman degradation or certain forms of mass spectroscopy.

Methods known in the art using combinations of these techniques may also be used. For example, in the HPLC-microscopy tandem mass spectrometry technique, proteolytic digestion is performed on a protein, and the resulting peptide mixture is separated by reversed-phase chromatographic separation. Tandem mass spectrometry is then performed and the data collected therefrom is analyzed. (Gatlin et al. (2000) Anal. Chem., 72:757-763). In another example, nondenaturing gel electrophoresis is combined with MALDI mass spectroscopy (Mathew et al. (2011) Anal. Biochem. 416: 135-137).

In some embodiments, the protein may be isolated from the sample using a reagent, such as antibody or peptide that specifically binds the protein, and then further analyzed to determine the presence or absence of the genetic variation using any of the techniques disclosed above.

Alternatively, the presence of the variant protein in a sample may be detected by immunoaffinity assays based on antibodies specific to proteins having genetic variations according to the present invention, that is, antibodies which specifically bind to the protein having the variation, but not to a form of the protein which lacks the variation. Such antibodies can be produced by any suitable technique known in the art. Antibodies can be used to immunoprecipitate specific proteins from solution samples or to immunoblot proteins separated by, e.g., polyacrylamide gels. Immunocytochemical methods can also be used in detecting specific protein variants in tissues or cells. Other well known antibody-based techniques can also be used including, e.g., enzyme-linked immunosorbent assay (ELISA), radioimmuno-assay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530.

### Identification of Additional Genetic Markers

The disclosed genetic markers are useful for identifying additional genetic markers associated with the development of AD. For example, the SNPs disclosed herein can be used to identify additional SNPs that are in linkage disequilibrium. Indeed, any SNP in linkage disequilibrium with a first SNP associated with AD will be associated with AD. Once the association has been demonstrated between a given SNP and AD, the discovery of additional SNPs associated with AD can be of great interest in order to increase the density of SNPs in this particular region.

Methods for identifying additional SNPs and conducting linkage disequilibrium analysis are well known in the art. For example, identification of additional SNPs in linkage disequilibrium with the SNPs disclosed herein can involve the steps of: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker.

### Additional Diagnostic Methods for Use in Combination

Detection of the disclosed genetic markers may be used in combination with one or more additional diagnostic approaches for identifying subjects as having AD or as having an increased risk for developing AD. For example, subjects can be screened for additional genetic markers in addition to the genetic markers disclosed herein. Cerebrospinal fluid from subjects may be analyzed for increased levels of amyloid beta or tau proteins that are characteristic of AD. Subjects can also be subjected to a mental status exam, such as the Mini Mental State Exam (MMSE) to assess memory, concentration, and other cognitive skills. Subjects can also be subjected to imaging procedures, such as a CT scan, an MRI, a SPECT scan or a PET scan to identify changes in brain structure or size indicative of Alzheimer's disease.

### Diagnosis, Prognosis and Treatment of Alzheimer's Disease

The invention provides methods for the diagnosis or prognosis of AD in a subject by detecting the presence in a sample from the subject of one or more genetic variations associated with AD as disclosed herein. In embodiments of the invention, the one or more genetic variation is in a gene selected from the genes coding for interleukin-6 receptor (IL6R), neurotrophic factor 4 (NTF4) and UNC5C, and any of the genes listed in Table 3. In some embodiments, the genetic variation is in genomic DNA that encodes a gene (or its regulatory region) wherein the gene is selected from the genes coding for interleukin-6 receptor (IL6R), neurotrophic factor 4 (NTF4) and UNC5C, and any of the genes listed in Table 3. In various embodiments, the genetic variation is a SNP, an allele, a haplotype, an insertion, or a deletion in one or more genes selected from the genes coding for interleukin-6 receptor (IL6R), neurotrophic factor 4 (NTF4) and UNC5C, and any of the genes listed in Table 3. In an embodiment, the genetic variation is a SNP that results in the amino acid substitution D358A in the amino acid sequence of IL6R (SEQ ID NO:1). In an embodiment, the genetic variation is a ‘C’ allele at rs2228145. In an embodiment, the genetic variation is a SNP that results in the amino acid substitution R206W in the amino acid sequence of NTF4 (SEQ ID NO:2). In an embodiment, the genetic variation is a ‘T’ allele at rs121918427. In an embodiment, the genetic variation is a SNP that results in the amino acid substitution T835M in the amino acid sequence of UNC5C (SEQ ID NO:3). In embodiments, the genetic variation is a SNP in a gene selected from those listed in Table 3. In embodiments the genetic variation is a SNP selected from rs12733578, rs4658945, rs1478161, rs1024591, rs7799010, rs10969475, and rs12961250. Any one or more of these genetic variations may be used in any of the methods of detection, diagnosis and prognosis described below.

In an embodiment, the invention provides a method for detecting the presence or absence of a genetic variation indicative of Alzheimer's disease (AD) in a subject, comprising: (a) contacting a sample from the subject with a reagent capable of detecting the presence or absence of a genetic variation in a gene selected from the genes encoding IL6R, NTF4 and UNC5C; and (b) determining the presence or absence of the genetic variation, wherein the presence of the genetic variation indicates that the subject is afflicted with, or at risk of developing, AD.

The reagent for use in the method may be an oligonucleotide, a DNA probe, an RNA probe, and a ribozyme. In some embodiments, the reagent is labeled. Labels may include, for example, radioisotope labels, fluorescent labels, bioluminescent labels or enzymatic labels. Radionuclides that can serve as detectable labels include, for example, I-131, I-123, I-125, Y-90, Re-188, Re-186, At-211, Cu-67, Bi-212, and Pd-109.

Also provided is a method for detecting a genetic variation indicative of Alzheimer's disease (AD) in a subject, comprising: determining the presence or absence of a genetic variation in a gene selected from the genes encoding IL6R, NTF4 and UNC5C in a biological sample from a subject, wherein the presence of the genetic variation indicates that the subject is afflicted with, or at risk of developing, AD. In various embodiments of the method, detection of the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism. In some embodiments, nucleic acids from the sample are amplified prior to determining the presence of the one or more genetic variation.

The invention further provides a method for diagnosing or prognosing AD in a subject, comprising: (a) contacting a sample from the subject with a reagent capable of detecting the presence or absence of a genetic variation in a gene selected from the genes encoding IL6R, NTF4 and UNC5C; and (b) determining the presence or absence of the genetic variation, wherein the presence of the genetic variation indicates that the subject is afflicted with, or at risk of developing, AD.

The invention further provides a method of diagnosing or prognosing AD in a subject, comprising: determining the presence or absence of a genetic variation in a gene selected from the genes encoding IL6R, NTF4 and UNC5C in a biological sample from a subject, wherein the presence of the genetic variation indicates that the subject is afflicted with, or at risk of developing, AD.

The invention also provides a method of diagnosing or prognosing AD in a subject, comprising: (a) obtaining a nucleic-acid containing sample from the subject, and (b) analyzing the sample to detect the presence of at least one genetic variation in a gene selected from the genes encoding IL6R, NTF4 and UNC5C, wherein the presence of the genetic variation indicates that the subject is afflicted with, or at risk of developing, AD.

In some embodiments, the method of diagnosis or prognosis further comprises subjecting the subject to one or more additional diagnostic tests for AD, for example, screening for one or more additional genetic markers, administering a mental status exam, or subjecting the subject to imaging procedures. In some embodiments, the method further comprises analyzing the sample to detect the presence of at least one additional genetic marker that is an APOE modifier, wherein the at least one additional genetic marker is in a gene selected from the gene encoding IL6R, the gene encoding NTF4, the gene encoding UNC5C, and a gene listed in Table 3.

It is further contemplated that any of the above methods may further comprise treating the subject for AD based on the results of the method. In some embodiments, the above methods further comprise detecting in the sample the presence of at least one APOE-ε4 allele. In an embodiment, the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

Also provided is a method of identifying a subject having an increased risk of earlier age of diagnosis of AD, comprising: (a) determining the presence or absence of a genetic variation in a gene selected from the genes encoding IL6R, NTF4 and UNC5C in a biological sample from a subject; and (b) determining the presence or absence of at least one APOE-ε4 allele, wherein the presence of the genetic variation and at least one APOE-ε4 allele indicates that the subject has an increased risk of earlier age of diagnosis of AD as compared to a subject lacking the presence of the genetic variation and at least one APOE-ε4 allele.

Also provided is a method of aiding prognosis of a subphenotype of AD in a subject, the method comprising detecting in a biological sample derived from the subject the presence of a genetic variant in a gene encoding IL6R, NTF4 or UNC5C. In an embodiment, the genetic variant is a SNP that results in the amino acid substitution D358A in the amino acid sequence of IL6R (SEQ ID NO:1), and the subphenotype of AD is characterized at least in part by increased levels of soluble IL6R in a biological sample derived from the subject as compared to one or more control subjects. In another embodiment, the genetic variation is a SNP that results in the amino acid substitution R206W in the amino acid sequence of NTF4 (SEQ ID NO:2), and the subphenotype of AD is characterized at least in part by decreased activation of TrkB in a biological sample derived from the subject as compared to one or more control subjects. In another embodiment, the genetic variation is a SNP that results in the amino acid substitution T835M in the amino acid sequence of UNC5C (SEQ ID NO:3), and the subphenotype of AD is characterized at least in part by increased apoptotic activity of UNC5C in a biological sample derived from the subject as compared to one or more control subjects.

The invention further provides a method of predicting the response of a subject to an AD therapeutic agent that targets IL6R, comprising detecting in a biological sample obtained from the subject a SNP that results in the amino acid substitution D358A in the amino acid sequence of IL6R (SEQ ID NO:1), wherein the presence of the SNP is indicative of a response to a therapeutic agent that targets IL6R. In an embodiment, the therapeutic agent is an IL6R antagonist or binding agent, for example, an anti-IL6R antibody.

The invention further provides a method of predicting the response of a subject to an AD therapeutic agent that targets TrkB, comprising detecting in a biological sample obtained from the subject a SNP that results in the amino acid substitution R206W in the amino acid sequence of NTF4 (SEQ ID NO:2), wherein the presence of the SNP is indicative of a response to a therapeutic agent that targets TrkB. In an embodiment, the therapeutic agent is a TrkB agonist, for example, a TrkB agonist antibody.

The invention further provides a method of predicting the response of a subject to an AD therapeutic agent that targets UNC5C, comprising detecting in a biological sample obtained from the subject a SNP that results in the amino acid substitution T835M in the amino acid sequence of UNC5C (SEQ ID NO:3), wherein the presence of the SNP is indicative of a response to a therapeutic agent that targets UNC5C. In an embodiment, the therapeutic agent targets the UNC5C death domain.

A biological sample for use in any of the methods described above may be obtained using certain methods known to those skilled in the art. Biological samples may be obtained from vertebrate animals, and in particular, mammals. In certain embodiments, a biological sample comprises a cell or tissue, such as cerebrospinal fluid, neural cells, or brain tissue. Variations in target nucleic acids (or encoded polypeptides) may be detected from a tissue sample or from other body samples such as cerebrospinal fluid, blood, serum, urine, sputum, saliva, mucosal scraping, lacrimal secretion, or sweat. By screening such body samples, a simple early diagnosis can be achieved for diseases such as AD. In addition, the progress of therapy can be monitored more easily by testing such body samples for variations in target nucleic acids (or encoded polypeptides). In some embodiments, the biological sample is obtained from an individual suspected of having AD.

Subsequent to the determination that a subject, or biological sample obtained from the subject, comprises a genetic variation disclosed herein, it is contemplated that an effective amount of an appropriate AD therapeutic agent may be administered to the subject to treat AD in the subject.

Also provided are methods for aiding in the diagnosis of AD in a mammal by detecting the presence of one or more variations in nucleic acid comprising a genetic variation in any one or more of the genes encoding IL6R, NTF4, or UNC5C as disclosed herein, or a gene listed in Table 3, according to the method described above.

In another embodiment, a method is provided for predicting whether a subject with AD will respond to a therapeutic agent by determining whether the subject comprises a variation in one or more of the genes encoding IL6R, NTF4, or UNC5C as disclosed herein, or a gene listed in Table 3, according to the method described above.

Also provided are methods for assessing predisposition of a subject to develop AD by detecting presence or absence in the subject of a variation in one or more of the genes encoding IL6R, NTF4, or UNC5C as disclosed herein, or a gene listed in Table 3.

Also provided are methods of sub-classifying AD in a mammal, the method comprising detecting the presence of a genetic variation in any one or more of the genes encoding IL6R, NTF4, or UNC5C as disclosed herein, or a gene listed in Table 3.

Also provided are methods of identifying a therapeutic agent effective to treat AD in a patient subpopulation, the method comprising correlating efficacy of the agent with the presence of a genetic variation at a nucleotide position corresponding to a SNP in any one or more of the genes encoding IL6R, NTF4, or UNC5C as disclosed herein, or a gene listed in Table 3.

Additional methods provide information useful for determining appropriate clinical intervention steps, if and as appropriate. Therefore, in one embodiment of a method of the invention, the method further comprises a clinical intervention step based on results of the assessment of the presence or absence of a variation in a gene associated with AD as disclosed herein. For example, appropriate intervention may involve prophylactic and treatment steps, or adjustment(s) of any then-current prophylactic or treatment steps based on genetic information obtained by a method of the invention.

As would be evident to one skilled in the art, in any method described herein, while detection of presence of a variation would positively indicate a characteristic of a disease (e.g., presence or subtype of a disease), non-detection of a variation would also be informative by providing the reciprocal characterization of the disease.

Still further methods include methods of treating AD in a mammal, comprising the steps of obtaining a biological sample from the mammal, examining the biological sample for the presence or absence of a variation as disclosed herein, and upon determining the presence or absence of the variation in said tissue or cell sample, administering an effective amount of an appropriate therapeutic agent to said mammal. Optionally, the methods comprise administering an effective amount of a targeted AD therapeutic agent to said mammal.

Also provided are methods of treating AD in a subject in whom a genetic variation is known to be present at a nucleotide position corresponding to a SNP in any one or more of the genes encoding IL6R, NTF4, or UNC5C as disclosed herein, or a gene listed in Table 3, the method comprising administering to the subject a therapeutic agent effective to treat the condition.

Also provided are methods of treating a subject having AD, the method comprising administering to the subject a therapeutic agent known to be effective to treat the condition in a subject who has a genetic variation at a nucleotide position corresponding to a SNP in any one or more of the genes encoding IL6R, NTF4, or UNC5C as disclosed herein, or a gene listed in Table 3.

Also provided are methods of treating a subject having AD, the method comprising administering to the subject a therapeutic agent previously shown to be effective to treat said condition in at least one clinical study wherein the agent was administered to at least five human subjects who each had a genetic variation at a nucleotide position corresponding to a SNP in any one or more of the genes encoding IL6R, NTF4, or UNC5C as disclosed herein, or a gene listed in Table 3. In one embodiment, the at least five subjects had two or more different SNPs in total for the group of at least five subjects. In one embodiment, the at least five subjects had the same SNP for the entire group of at least five subjects.

Also provided are methods of treating an AD subject who is of a specific AD patient subpopulation comprising administering to the subject an effective amount of a therapeutic agent that is approved as a therapeutic agent for said subpopulation, wherein the subpopulation is characterized at least in part by association with genetic variation at a nucleotide position corresponding to a SNP in any one or more of the genes encoding IL6R, NTF4, or UNC5C as disclosed herein, or a gene listed in Table 3.

In one embodiment, the subpopulation is of European ancestry. In one embodiment, the invention provides a method comprising manufacturing an AD therapeutic agent, and packaging the agent with instruction to administer the agent to a subject who has or is believed to have AD and who has a genetic variation at a position corresponding to a SNP in any one or more of the genes encoding IL6R, NTF4, or UNC5C as disclosed herein, or a gene listed in Table 3.

Also provided are methods for selecting a patient suffering from AD for treatment with an AD therapeutic agent comprising detecting the presence of a genetic variation at a nucleotide position corresponding to a SNP in any one of the genes encoding IL6R, NTF4, or UNCSC as disclosed herein, or a gene listed in Table 3.

A therapeutic agent for the treatment of AD may be incorporated into compositions, which in some embodiments are suitable for pharmaceutical use. Such compositions typically comprise the peptide or polypeptide, and an acceptable carrier, for example one that is pharmaceutically acceptable. A “pharmaceutically acceptable carrier” includes any and all solvents, dispersion media, coatings, antibacterial and antifungal agents, isotonic and absorption delaying agents, and the like, compatible with pharmaceutical administration (Gennaro, Remington: The science and practice of pharmacy. Lippincott, Williams & Wilkins, Philadelphia, Pa. (2000)). Examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

A therapeutic agent of the invention (and any additional therapeutic agent for the treatment of AD) can be administered by any suitable means, including parenteral, intrapulmonary, intrathecal and intranasal, and, if desired for local treatment, intralesional administration. Parenteral infusions include, e.g., intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Certain embodiments of the invention provide for the AD therapeutic agent to traverse the blood-brain barrier. Several art-known approaches exist for transporting molecules across the blood-brain barrier, including, but not limited to, physical methods, lipid-based methods, and receptor and channel-based methods.

Physical methods of transporting the AD therapeutic agent across the blood-brain barrier include, but are not limited to, circumventing the blood-brain barrier entirely, or by creating openings in the blood-brain barrier. Circumvention methods include, but are not limited to, direct injection into the brain (see e.g., Papanastassiou et al., Gene Therapy 9: 398-406 (2002)) and implanting a delivery device in the brain (see e.g., Gill et al., Nature Med. 9: 589-595 (2003); and Gliadel Wafers™, Guildford Pharmaceutical). Methods of creating openings in the barrier include, but are not limited to, ultrasound (see e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see e.g., U.S. Pat. Nos. 5,112,596, 5,268,164, 5,506,206, and 5,686,416), and transfection of neurons that straddle the blood-brain barrier with vectors containing genes encoding the antibody or fragment thereof (see e.g., U.S. Patent Publication No. 2003/0083299).

Lipid-based methods of transporting the AD therapeutic agent across the blood-brain barrier include, but are not limited to, encapsulating the AD therapeutic agent in liposomes that are coupled to antibody binding fragments that bind to receptors on the vascular endothelium of the blood-brain barrier (see e.g., U.S. Patent Application Publication No. 20020025313), and coating the AD therapeutic agent in low-density lipoprotein particles (see e.g., U.S. Patent Application Publication No. 20040204354) or apolipoprotein E (see e.g., U.S. Patent Application Publication No. 20040131692).

Receptor-based methods of transporting the AD therapeutic agent across the blood-brain barrier include, but are not limited to, conjugation of the AD therapeutic agent to ligands that recognize receptors expressed at the blood-brain barrier, resulting in their being carried across the blood-brain barrier after receptor-mediated transcytosis (Gabathuler (2010) Neurobiology of Disease 37; 48-57). These ligands include but are not limited to ligands for brain capillary endothelial receptors such as a monoclonal antibody to the transferrin receptor or to the insulin receptor, histones, biotin, folate, niacin, panthothenic acid, or glycopeptides.

Effective dosages and schedules for administering AD therapeutic agents may be determined empirically, and making such determinations is within the skill in the art. Single or multiple dosages may be employed. When in vivo administration of an AD therapeutic agent is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. No. 4,657,760; 5,206,344; or 5,225,212.

It is contemplated that yet additional therapies may be employed in the methods. The one or more other therapies may include but are not limited to, administration of an additional AD therapeutic agent, such as a cholinesterase inhibitor, memantine, an anti-agitation medication, an anti-depressive, an anxiolytic, or a compound targeting amyloid precursor protein, amyloid beta, amyloid plaques, or any of the enzymes that cleave amyloid precursor protein including, but not limited to alpha-secretase, beta-secretase, and gamma-secretase, and the like.

### Kits

For use in the applications described or suggested herein, kits or articles of manufacture are also provided. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising a genetic variant associated with AD as disclosed herein. Where the kit utilizes nucleic acid hybridization to detect a target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label.

In other embodiments, the kit may comprise a labeled agent capable of detecting a polypeptide comprising a genetic variant associated with AD as disclosed herein. Such agent may be an antibody which binds the polypeptide. Such agent may be a peptide which binds the polypeptide. The kit may comprise, for example, a first antibody (e.g., attached to a solid support) which binds to a polypeptide comprising a genetic variant as disclosed herein; and, optionally, a second, different antibody which binds to either the polypeptide or the first antibody and is conjugated to a detectable label.

Kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above. Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

### Methods of Marketing

The invention herein also encompasses a method for marketing the disclosed methods of diagnosis or prognosis of AD comprising advertising to, instructing, and/or specifying to a target audience, the use of the disclosed methods.

Marketing is generally paid communication through a non-personal medium in which the sponsor is identified and the message is controlled. Marketing for purposes herein includes publicity, public relations, product placement, sponsorship, underwriting, and the like. This term also includes sponsored informational public notices appearing in any of the print communications media.

The marketing of the diagnostic method herein may be accomplished by any means. Examples of marketing media used to deliver these messages include television, radio, movies, magazines, newspapers, the internet, and billboards, including commercials, which are messages appearing in the broadcast media.

The type of marketing used will depend on many factors, for example, on the nature of the target audience to be reached, e.g., hospitals, insurance companies, clinics, doctors, nurses, and patients, as well as cost considerations and the relevant jurisdictional laws and regulations governing marketing of medicaments and diagnostics. The marketing may be individualized or customized based on user characterizations defined by service interaction and/or other data such as user demographics and geographical location.

The following are examples of the methods and compositions of the invention. It is understood that various other embodiments may be practiced, given the general description provided above.

### EXAMPLES

### Example 1

### APOE Modifier Screen

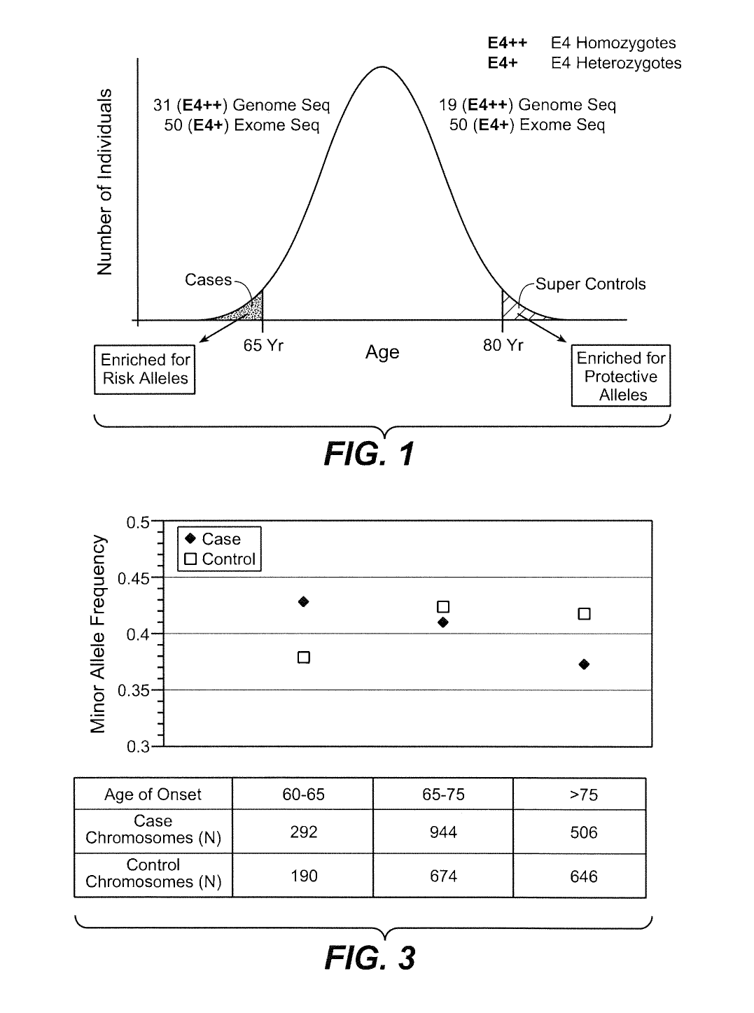

A study was designed to identify variants that modify the effect of APOE on the development of Alzheimer's disease (AD). The study design is illustrated in FIG. 1. DNA isolated from subjects under 65 years of age and having AD, thus presumably enriched for risk alleles (the “cases”), was compared to DNA isolated from subjects over 75 or 80 years of age without AD and with normal cognition by neurologic testing, thus presumably enriched for protective alleles (the “supercontrols”). All subjects were either homozygous (E4/E4) or heterozygous (E3/E4) for the APOE E4 allele, United States residents of European descent, and were obtained from the National Cell Repository of Alzheimer's Disease (NCRAD). As shown in Table 1, the cases for Cohort 1 included a total of 31 unrelated E4/E4 homozygotes and 50 E3/E4 Alzhiemer's cases with an age of dementia onset <65 and >55 years of age. For approximately one-third of the cases a diagnosis of AD was confirmed by autopsy. The supercontrols for Cohort 1 included 19 E3/E4 heterozygotes over 80 years of age and 50 E4/E4 homozygotes over 75 years of age. The controls all had a Clinical Dementia Rating (CDR) scale equal to 0, indicating no evidence of cognitive impairment at the last visit. The APOE allele of the samples was confirmed by whole genome sequencing (for heterozygotes) or by exome sequencing (for homozygotes).

TABLE 1
Cohort 1*

| APOE alleles | Cases (N) | Supercontrols (N) |
|---|---|---|
| APOE-ε4/APOE-ε4 | 31 | 19 |
| APOE-ε4/APOE-ε3 | 50 | 50 |

*Samples from the National Cell Repository for Alzheimer's Disease (NCRAD), which receives government support under a cooperative agreement grant (U24 AG21886) awarded by the National Institute on Aging (NIA), were used in this study. We thank contributors, including the Alzheimer's Disease Centers who collected samples used in this study, as well as patients and their families, whose help and participation made this work possible.

### Common Modifiers of APOE Risk

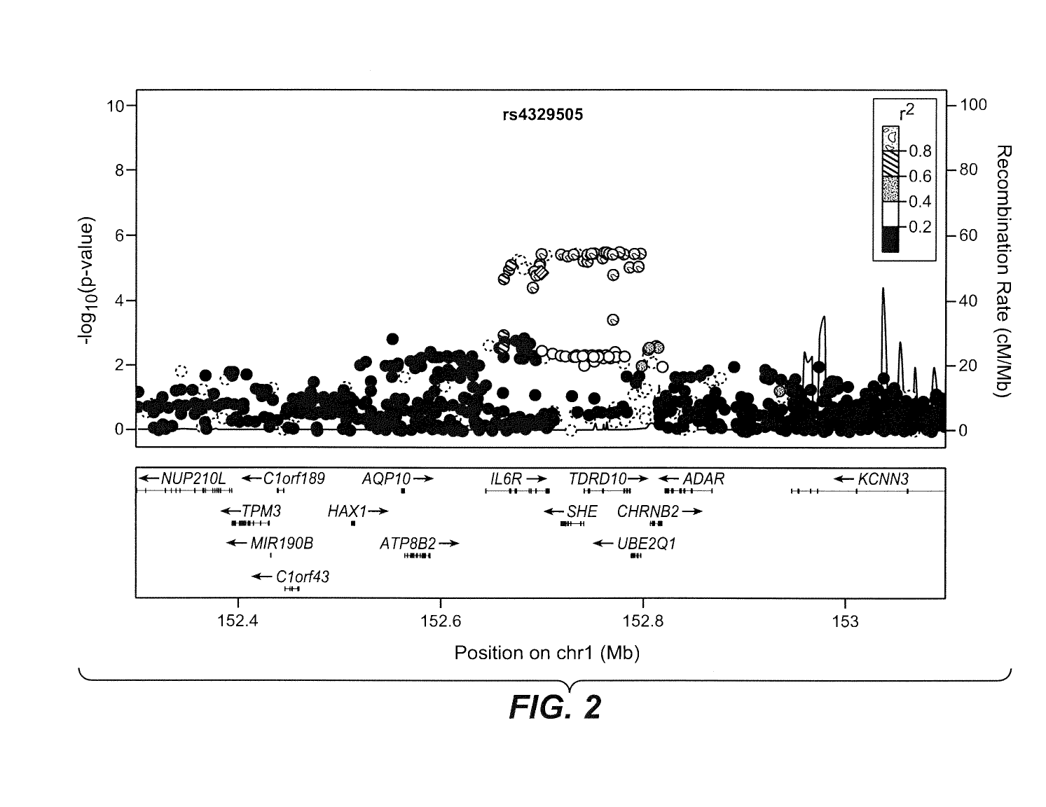

A genome wide association scan was performed in Cohort 1 to identify common variants that modify APOE risk. Subjects in Cohort 1 were genotyped using the Illumina 1M SNP array. Quality control for the genotyping data was performed as described in Gateva et al. (2009) Nat. Genet. 2009 November; 41(11):1228-1233. Cohort 1 (Table 1) was used for the discovery phase. Common variants in the IL6R/SHE/TDR10 region on human chromosome 1 showed significant association in the 81 E4+ cases versus the 68 E4+ controls of cohort 1 (FIG. 2).

A replication data set was obtained from the database of Genotypes and Phenotypes (dbGAP) available at the website of the National Center for Biotechnology Information (NCBI) for the National Institute on Aging—Late Onset Alzheimer's Disease Family Study: Genome-Wide Association Study for Susceptibility Loci (dbGAP study ID: phs000168.v1.p1). The NIA/LOAD study consisted of 932 AD cases and 836 controls of European-American ancestry genotyped for the Illumina 610K SNP array. Two hundred E4 heterozygote and homozygotye cases with an age of diagnosis <65 and 144 E4 heterozygote and homozygotye controls with an age at last visit of >=75 years were selected.

As shown in Table 2, a SNP (rs2228154) in the gene encoding IL6R was confirmed to be significantly associated with AD in both the discovery and replication cohorts. The variant of SNP rs2228145 having a C at the polymorphic site (the “C allele”), was preferentially found in AD cases as compared to controls. This allele comprises the amino acid substitution D358A in IL6R.

TABLE 2

| | | | | |
|---|---|---|---|---|
| Chromosome | SNP | Allele | Odds Ratio | P |

Discovery: 81 < 65 year old subject ALZ E4+ cases vs.
68 > 80 year old subject E4+ controls

| | | | | |
|---|---|---|---|---|
| 1 | rs2228154 | C | 1.738 | 0.0087 |

C allele: 47% in cases, 31% in controls

Replication: 200 < 65 year old subject ALZ E4+ cases vs.
144 > 75 year old subject E4+ controls

| | | | | |
|---|---|---|---|---|
| 1 | rs2228154 | C | 1.642 | 0.0017 |

C allele: 46% in cases, 33% in controls
The meta P value was 4.7 × 10−5.

The distribution of the A358 variant allele of IL6R was further examined in the 932 unselected AD cases and 836 controls from the NIA/LOAD study. The clinical assessment and genotyping of APOE polymorphisms in the NIA/LOAD subjects was described in Lee et al., (2008) Arch Neurol. 65: 1518-1526. FIG. 3 shows the frequency of the T allele of rs4129267, a proxy of the C allele of rs2228145, in unselected AD cases and controls from the NIA/LOAD study, as stratified by age of onset in AD cases and age in controls. The A358 variant allele was present more frequently in the earlier onset cases as compared to controls, but less frequently in the later onset cases compared to controls, consistent with a disease modifying variant.

The interleukin-6 receptor (IL6R) is the receptor for the cytokine interleukin 6 (IL-6), a potent pleiotropic cytokine that regulates cell growth and differentiation and plays an important role in the immune response. IL6R A358 is a common variant allele associated with increased serum IL6R levels (Galicia et al. (2004) Genes & Immunity 5:513; Marinou et al. (2010) Ann. Rheum. Dis. 69: 1191). The A358 variant allele has been associated with decreased CRP circulating levels and decreased risk of coronary heart disease (Elliott et al. (2009) JAMA 302: 37-48), as well as increased asthma risk (Ferreira et al. (2011) Lancet 378: 1006-1014).

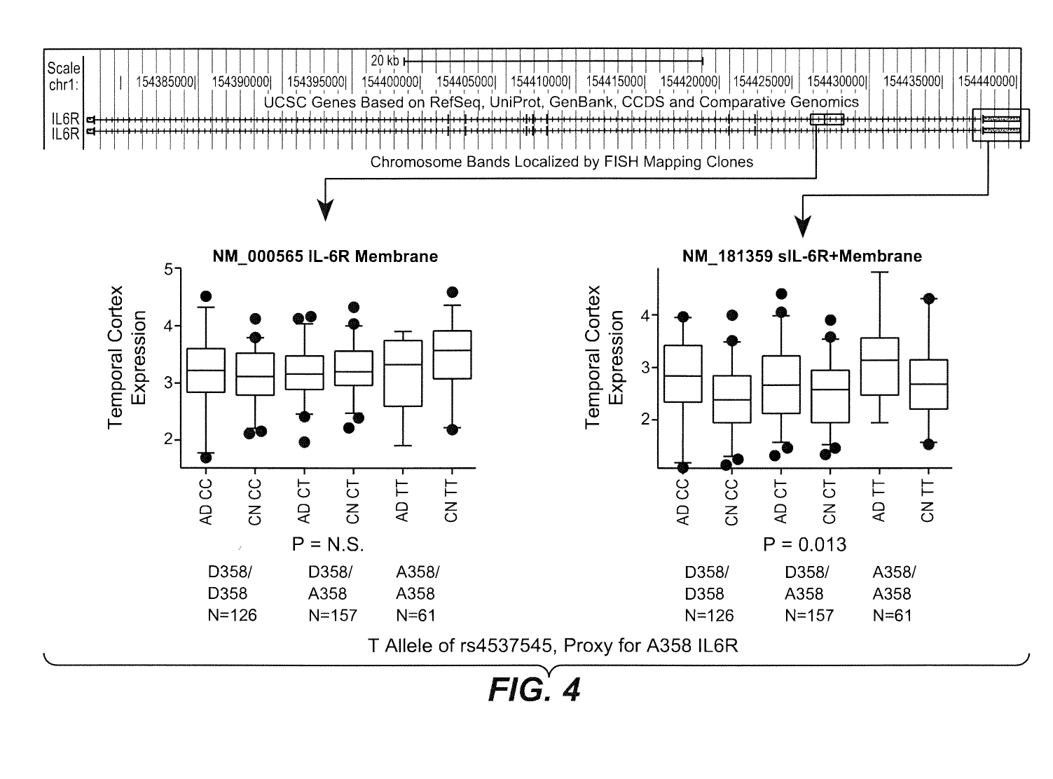

To examine if IL6R mRNA expression is elevated in AD and/or affected by the genotype at position 358, data from the TGEN project (Webster et al. (2009) Am. J. Hum. Genet. 84: 445-458) was analyzed to compare the expression levels of both membrane bound and soluble IL6R in the brains of subjects with AD as compared to controls. Using a probe that detects only the membrane bound form of IL6R(NM_{—}000565), no enrichment in AD or by genotype at position 358 was observed. However, using a probe that captures both the membrane bound and sIL6R(NM_{—}181359) mRNA, significant enrichment in AD cases as compared to controls was observed in the temporal region of the brain and by genotype at position 358 (FIG. 4).

In addition, to the IL6R region, the regions listed in Table 3 showed significant association in Cohort1 and the NIA/LOAD dataset, suggesting these loci may be additional common modifiers of APOE risk.

TABLE 3
Regions associated with APOE risk in Cohort1 and the NIA/LOAD study

| | | Cohort 1 (81 case vs | | | | NIA/LOAD (200 case vs | | | |
|---|---|---|---|---|---|---|---|---|---|
| | | 68 control) | | | | 144 control) | | | |
| SNP | GENE | Freq_A | Freq_U | OR | P | Freq_A | Freq_U | OR | P |
| rs12733578 | INPP5B | 0.19 | 0.36 | 0.45 | 0.00044 | 0.28 | 0.38 | 0.66 | 0.013 |
| rs4658945 | DISC1 | 0.37 | 0.24 | 1.83 | 0.0058 | 0.30 | 0.22 | 1.46 | 0.037 |
| rs1478161 | OTOLIN1 | 0.21 | 0.34 | 0.48 | 0.0023 | 0.26 | 0.34 | 0.70 | 0.035 |
| rs1024591 | STAG3L4 | 0.48 | 0.34 | 1.82 | 0.0032 | 0.49 | 0.39 | 1.53 | 0.0093 |
| rs7799010 | UBE3C/ | 0.50 | 0.37 | 1.78 | 0.0074 | 0.48 | 0.40 | 1.40 | 0.035 |
| | MINX1 | | | | | | | | |
| rs10969475 | LINGO2/ | 0.52 | 0.37 | 1.87 | 0.0024 | 0.50 | 0.41 | 1.44 | 0.026 |
| | ACO1 | | | | | | | | |
| rs12961250 | MRLC2 | 0.40 | 0.27 | 1.99 | 0.0023 | 0.41 | 0.33 | 1.38 | 0.050 |
| rs225359 | TFF1 | 0.25 | 0.43 | 0.44 | 0.00022 | 0.30 | 0.41 | 0.64 | 0.0054 |

### Rare Variants in the APOE Modifier Screen

Neurotrophin 4 (NTF4)

In addition to the common variants disclosed above, the APOE modifier screen also resulted in the identification of rare variants associated with AD. These rare variants, having less than 2% allele frequency in the overall population, included the R206W variant of NTF4. The R206W variant of NTF4 was found in 2 of 78 (2.6%) of AD cases and 0 of 67 (0.0%) of supercontrols. In addition, the R206W variant was not observed in 1300 exome-sequenced European-Americans (P=1.87×10⁻⁹) who did not have AD at the time of sample collection, using data obtained from the NHLBI Exome Sequencing Project (ESP) exome variant server.

The R206W variant of NTF4 results from a C to T substitution at the site of SNP rs121918427 on chromosome 19. Neurotrophin 4 is a member of a family of neurotrophic factors, the neurotrophins (NTs), that control survival and differentiation of mammalian neurons. The neurotrophins are responsible for the maintenance, proliferation and differentiation of subsets of neurons bearing specific tyrosine kinase receptors, the Trks. Trk activation by NTs promotes neuron survival through the negation of programmed cell death (Robinson et al. (1999) Protein Sci. 8: 2589-2597). NTF4 promotes the survival of peripheral and sympathetic neurons, and activates both Trk and TrkB (Berkemeier et al. (1991) Neuron 7: 857-866).

The NTF4 R206W variant has previously been reported to be overrepresented in subjects with glaucoma as compared to controls. ((Passuto et al. (2009) Am. J. Hum. Genet. 85: 447-456); Liu et al. (2010) Am. J. Hum. Genet. 86: 498-499). The altered residue is highly conserved among orthologs in chimpazee, dog, mouse and rat, and is located in the TrkB binding site. The variant protein has reduced ability to activate TrkB, and demonstrates impaired function in neurite outgrowth. The variant protein is thus predicted to have an effect on neuronal survival. (Passuto et al., supra).

This newly identified association of this impaired-function R206W variant of NTF4 with earlier-onset AD in APOE4 carriers suggests that activation of the NTF4 pathway may be protective against the development of AD and that agonists of the TrkB receptor may be potential therapeutics for treatment of AD.

### Example 2

### Family Based Screening

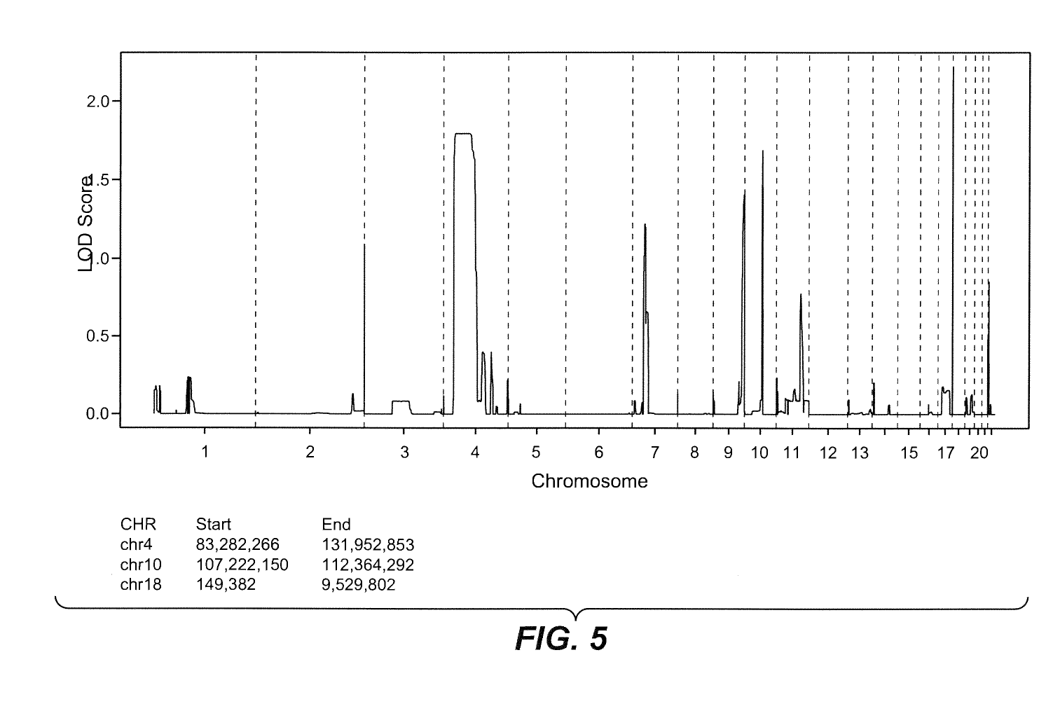

The LO1 pedigree was obtained via collaboration with Alison Goate (Washington University). The LO1 pedigree showed a pattern suggesting dominant inheritance of AD. The proband was one of five siblings, two of whom also had AD, while the AD status of another sibling was undetermined. The mother of the proband had AD, and the father did not. A half-sibling of the proband, the child of the proband's father by another spouse, did not have AD. Of the children of the proband and siblings, four had AD. The age of AD onset in family members ranged from 58 to 87. Nonparametric linkage analysis was carried out using genotype data collected using the Illumina Linkage Array obtained from 16 members of the LO1 pedigree. The NPL linkage was run using MERLIN software using a QCed dataset. The results of nonparametric linkage analysis in the LO1 pedigree are shown in FIGS. 5, and 3 regions with an NPL lod score >1.5 were observed. To identify potential causal alleles within the 3 linkage intervals, exome sequencing was carried out for the proband (Illumina shot read technology), and analysis was restricted to NPL linkage peaks having an LOD score greater than 1.5. The resultant 4,153 variants were ranked based upon novelty (defined as presence in dbSNP or 1000 genome project data), heterozygosity and putative function. The genotype of another AD case, a niece of the proband, was determined using complete genome sequencing (CGI), and the presence or absence of the top five ranked variants was determined. A single variant was identified by this process, and is located in chromosome 4. The presence or absence of this variant was determined for 19 members of the LO1 pedigree, including the proband, the proband's mother, three siblings, and the children of the proband and all siblings. Of the eleven carriers, eight had AD, while the disease status of one other was unknown. The two remaining carriers did not have AD, but were less than 75 years old. None of the eight family members lacking the variant had AD.

The variant was found to be a G to A substitution in chromosome 4, resulting in the amino acid substitution T835M in the gene encoding UNC5C. UNC5C is a member of the UNC5 family of netrin receptors, and is a receptor for netrin 1. UNC5C is highly expressed in hippocampal neurons. UNC5A, B and C mediate the chemorepulsive effect of netrin 1 on specific axons. These receptors are also dependence receptors which induce apoptosis when unbound to their netrin 1 ligand. The pro-apoptotic activity of these receptors depends upon cleavage of the receptors by caspase and the presence of a conserved death domain in the C-terminus of the intracellular domain.

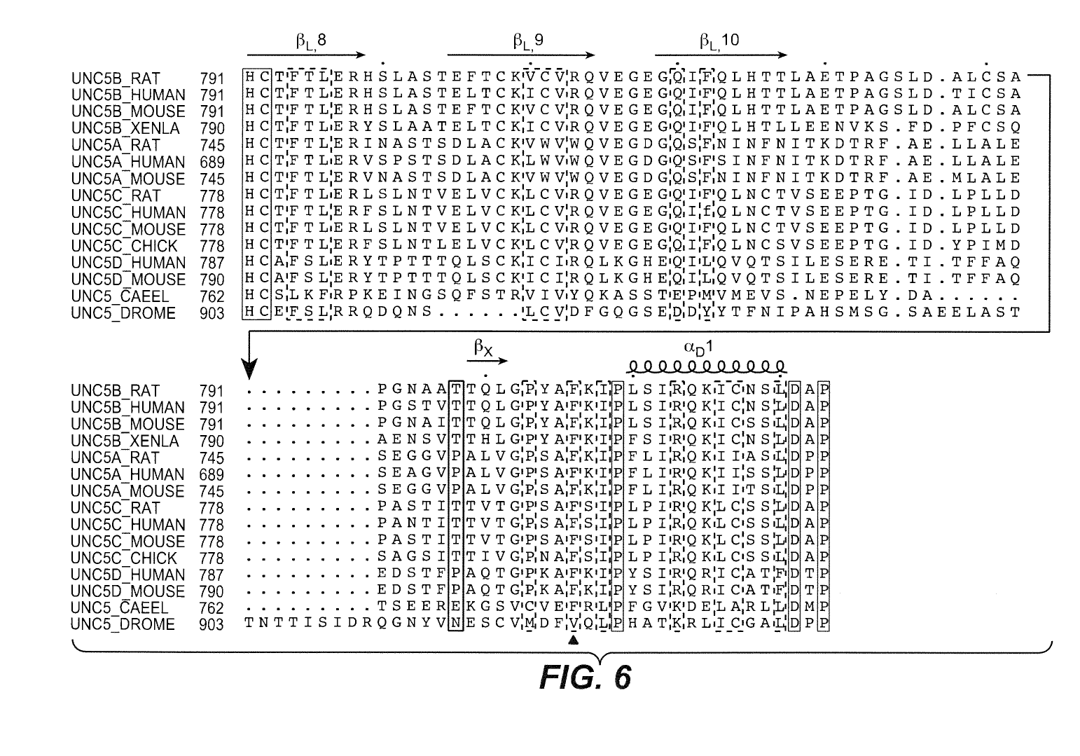

The SIFT program (Ng and Henikoff (2003) Nucleic Acids Res. 31: 3812-3814) was used to predict whether this amino acid substitution is expected to affect protein function. A SIFT score of less than 0.05 is indicative of a deleterious substitution. The SIFT score of the T835M variant was 0.01, indicating that this variant has a high likelihood of being deleterious. An alignment to other UNC5 family members (FIG. 6) shows that this variant is present in a conserved motif. Based upon the structure of the UNC5 proteins, this variant is in a hinge region between the death domain and the ZU5 domain, a region that interacts with downstream regulators of apoptosis (Williams et al. (2003) J. Biol. Chem. 278: 17483-17490). Given the function of UNC5C as a netrin receptor and its high expression in hippocampal neurons, the T835M variant may affect UNC5C signaling such that the death domain of UNC5C is preferentially found in an open, activated state, resulting in increased pro-apoptotic signaling and neuronal cell death. This newly identified association of the T835M variant of UNC5C with AD suggests that blocking the aberrant apoptotic signaling of this UNC5C variant may be a potential therapeutic approach for the treatment of AD.

The data from the APOE modifier screen described in Example 1 was assessed for the presence of the T385M variant of UNC5C. The T835M variant of UNC5C was observed in 2/78 AD cases and 1/67 controls. Genotyping of >6,000 additional controls established a population allele frequency of T825M in European-American populations to be 0.00071 (9/6315 individuals heterozygous for T835M) (Table 4), and an AD case frequency of 0.013 (P=1.5×10⁻⁷). This data suggests that T835M is a rare variant that increases risk of AD.

TABLE 4

| | | Allele | | Allele |
|---|---|---|---|---|
| Study | AD cases | frequency | Controls | frequency |
| APOE modifier screen | 2/78 | 0.013 | 1/67 | 0.0074 |
| NF1, MADGC | | | 0/200 | 0.0 |
| AREDS | | | 6/2763 | 0.00011 |
| Colorectal Cancer | | | 0/235 | 0.0 |
| EVS (WashU) | | | 1/1350 | 0.00037 |
| NYCP | | | 1/1700 | |
| Combined | 2/78 | 0.013 | 9/6315 | 0.000712 |

### Example 3

### Association of A358 with Increased Soluble IL6R Levels

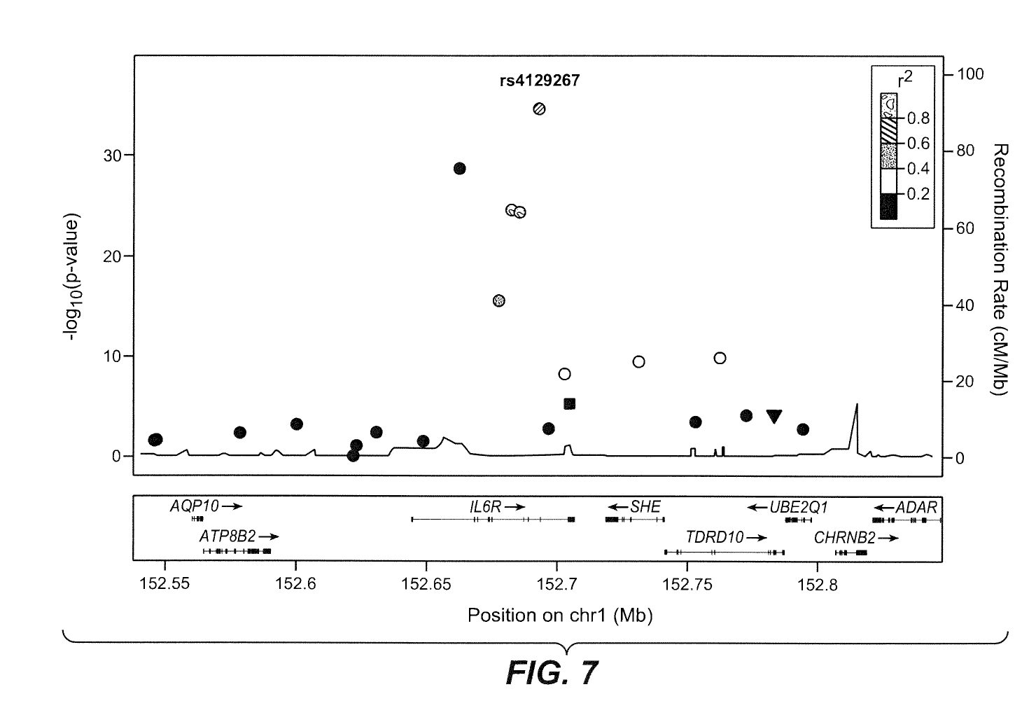

Tests were performed for association between soluble IL6R (sIL6R) levels in cerebrospinal fluid (CSF) and the genotypes at SNPs in the IL6R genic region in data for 291 samples from the Alzheimer's Disease Neuroimaging Initiative (ADNI; Weiner, M. W. et al. (2010) Alzheimer's & Dementia 6: 202-211). Subjects were genotyped using Illumina's Human610Quad genome-wide SNP array, and sIL6R was measured using an immunoassay panel based on Luminex immunoassay technology developed by Rules Based Medicine (MyriadRBM). At each SNP, a linear regression of log(sIL6R) was performed on the SNP genotype coded in an additive manner (0, 1, or 2 mutant alleles), and the null hypothesis that the effect size of the genotype was zero was tested. Variants in the IL6R gene showed significant association with increased sIL6R levels in CSF (FIG. 7), with the SNP rs4129267, a proxy of rs2228145, showing the strongest association.

As discussed above, the variant of SNP rs2228145 results in the amino acid substitution D358A in IL6R. As indicated in Table 5, the IL6R genotype at position 358 was correlated with soluble IL6R levels in CSF, with the presence of the A358 variant allele being associated with higher levels of CSF sIL6R.

TABLE 5

| IL6R Genotype | CSF sIL6R (mean) | N |
|---|---|---|
| D/D 358 | 0.85 ng/ml | 99 |
| D/A 358 | 1.19 ng/ml | 138 |
| A/A 358 | 1.43 ng/ml | 38 |

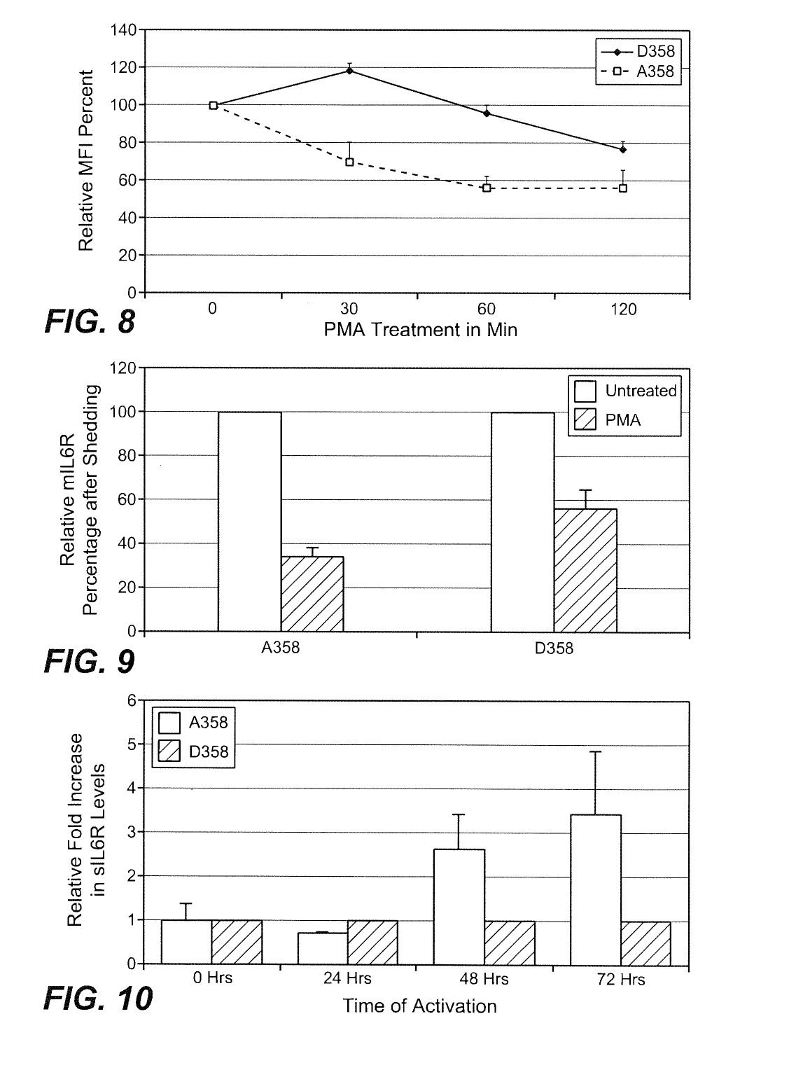

The effect of the presence of the A358 variant in IL6R on IL6R shedding was examined both in vitro and in vivo. For the in vitro experiments, 293T cells were transfected with D358 or A358 constructs of IL6R. 48 hours after transfection, the media was changed and cells were treated with 100 nM phorbol myristate acetate (PMA) for 0, 30, 60 and 120 minutes. Cells were harvested after the treatment and stained with an IL6R-PE antibody (BD Pharmingen, Cat. No-551850). Membrane-bound IL6R was analyzed by FACS. FIG. 8 shows the percent mean fluorescence intensity (MFI) relative to the 0 minutes time point at successive time points after treatment with PMA. This data demonstrates that the A358 variant leads to increased shedding of IL6R in 293T cells since the amount of cell-bound IL6R detected decreased notably in the variant A358-containing samples in contrast to that detected in the wild-type D358-containing samples.

Experiments were also carried out to determine whether the presence of the A358 variant allele in IL6R also leads to increased shedding of IL6R in primary T cells. Healthy human volunteers were genotyped for IL6R SNP rs2228145 by real-time quantitative PCR using the TaqMan SNP Genotyping Assay, Assay ID C_{—}16170664_{—}10 from Applied Biosystems. Peripheral blood mononuclear cells (PBMCs) were obtained by Ficol gradient from a pair of homozygous donors (one with each genotype AA and CC) that were age, gender and ethnicity matched. CD4⁺T cells were purified from PBMCs by negative selection using the EasySep CD4⁺T cells enrichment kit (Cat. No. 19052) from STEMCELL Technologies as recommended by the manufacturer. The CD4⁺ T cells were then cultured for 72 hours in RPMI 1640+10% FBS+2-mercaptoethanol and treated with 100 nM PMA for 60 min. Cells were harvested soon after the treatment and stained with the IL6R-PE antibody (BD Pharmingen, Cat. No-551850). The membrane bound IL6R was analyzed by FACS. FIG. 9 shows that the membrane bound fraction of IL6R was lower in CD4⁺T cells carrying IL6R with the A358 mutation as opposed to the wild-type D358 IL6R after activation by PMA, indicating increased shedding in A358 cells.

In another experiment, CD4⁺T cells were activated by plate bound anti hCD3 (BD Pharmingen, Cat.No-555329, 10 mg/ml) and anti hCD28 (BD-Cat No-555725, 5 mg/ml) or an isotopic control (BD Pharmingen, Cat No 554721-15 mg/ml). Cells were then harvested after 24, 48 and 72 hours for total RNA extraction and the supernatant was collected to determine the sIL6R levels by ELISA using the Human IL-6 R alpha Quantikine ELISA Kit (R&D Systems, Cat. No. DR600). FIG. 10 shows the fold increase in soluble IL6R for A358, relative to D358, at each time point. While the amount of soluble IL6R remained roughly constant over time for D358, it increased four-fold for A358 over the course of the experiment.

What is claimed is:

1. A method for detecting the presence or absence of a genetic variation indicative of Alzheimer's disease (AD) in a subject, comprising:
(a) contacting a sample from the subject with a reagent capable of detecting the presence or absence of a genetic variation in a gene selected from the genes encoding IL6R, NTF4 and UNC5C, or a gene product thereof; and
(b) determining the presence or absence of the genetic variation, wherein the presence of the genetic variation indicates that the subject is afflicted with, or at risk of developing, AD.

2. The method of claim 1, wherein the at least one genetic variation is a single nucleotide polymorphism (SNP), an allele, a haplotype, an insertion, or a deletion.

3. The method of claim 2, wherein the genetic variation is a SNP.

4. The method of claim 3 wherein the genetic variation is a SNP that results in the amino acid substitution D358A in the amino acid sequence of IL6R (SEQ ID NO:1).

5. The method of claim 4, wherein the genetic variation is a ‘C’ allele at rs2228145.

6. The method of claim 3 wherein the genetic variation is a SNP that results in the amino acid substitution R206W in the amino acid sequence of NTF4 (SEQ ID NO:2).

7. The method of claim 6 wherein the genetic variation is a ‘T’ allele at rs121918427.

8. The method of claim 3 wherein the genetic variation is a SNP that results in the amino acid substitution T835M in the amino acid sequence of UNC5C (SEQ ID NO:3).

9. The method of claim 8 wherein the genetic variation is a SNP that substitutes G for A in the codon encoding for the amino acid at position 835 of UNC5C (SEQ ID NO:3).

10. The method of claim 1 wherein the reagent is selected from an oligonucleotide, a DNA probe, an RNA probe, and a ribozyme.

11. The method of claim 10 wherein the reagent is labeled.

12. The method of claim 1 wherein the at least one genetic variation is an amino acid substitution, insertion or deletion in a protein selected from IL6R, NTF4 and UNC5C.

13. The method of claim 12 wherein the at least one genetic variation is an amino acid substitution selected from D358A in the amino acid sequence of IL6R (SEQ ID NO:1), R206W in the amino acid sequence of NTF4 (SEQ ID NO:2), and T835M in the amino acid sequence of UNC5C (SEQ ID NO:3).

14. The method of claim 12 wherein the reagent is an antibody that specifically binds to a protein comprising the genetic variation.

15. The method of claim 1, wherein the sample is selected from one of cerebrospinal fluid, blood, serum, sputum, saliva, mucosal scraping, tissue biopsy, lacrimal secretion, semen, or sweat.

16. The method of claim 1, further comprising treating the subject for AD based on the results of step (b).

17. The method of claim 1 further comprising detecting in the sample the presence of at least one APOE-ε4 allele.

18. The method of claim 17, wherein the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

19. A method for detecting a genetic variation indicative of Alzheimer's disease (AD) in a subject, comprising:
determining the presence or absence of a genetic variation in a gene selected from the genes encoding IL6R, NTF4 and UNC5C, or a gene product thereof, in a biological sample from a subject, wherein the presence of the genetic variation indicates that the subject is afflicted with, or at risk of developing, AD.

20. The method of claim 19, wherein the at least one genetic variation is a single nucleotide polymorphism (SNP), an allele, a haplotype, an insertion, or a deletion.

21. The method of claim 20, wherein the genetic variation is a SNP.

22. The method of claim 21 wherein the genetic variation is a SNP that results in the amino acid substitution D358A in the amino acid sequence of IL6R (SEQ ID NO:1).

23. The method of claim 22, wherein the genetic variation is a ‘C’ allele at rs2228145.

24. The method of claim 21 wherein the genetic variation is a SNP that results in the amino acid substitution R206W in the amino acid sequence of NTF4 (SEQ ID NO:2).

25. The method of claim 24 wherein the genetic variation is a ‘T’ allele at rs121918427.

26. The method of claim 21 wherein the genetic variation is a SNP that results in the amino acid substitution T835M in the amino acid sequence of UNC5C (SEQ ID NO:3).

27. The method of claim 26 wherein the genetic variation is a SNP that substitutes G for A in the codon encoding for the amino acid at position 835 of UNC5C (SEQ ID NO:3).

28. The method of claim 19 wherein the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism.

29. The method of claim 25 wherein nucleic acids from the sample are amplified prior to determining the presence of the one or more genetic variation.

30. The method of claim 19 wherein the at least one genetic variation is an amino acid substitution, insertion or deletion in a protein selected from IL6R, NTF4 and UNC5C.

31. The method of claim 30 wherein the at least one genetic variation is an amino acid substitution selected from D358A in the amino acid sequence of IL6R (SEQ ID NO:1), R206W in the amino acid sequence of NTF4 (SEQ ID NO:2), and T835M in the amino acid sequence of UNC5C (SEQ ID NO:3).

32. The method of claim 19 wherein the presence of the one or more genetic variation is carried out by a process selected from electrophoresis, chromatography, mass spectroscopy, proteolytic digestion, protein sequencing, immunoaffinity assay, or a combination thereof.

33. The method of claim 25 wherein proteins from the sample are purified using antibodies or peptides that bind the proteins prior to determining the presence of the one or more genetic variation.

34. The method of claim 19, wherein the sample is selected from one of cerebrospinal fluid, blood, serum, sputum, saliva, mucosal scraping, tissue biopsy, lacrimal secretion, semen, or sweat.

35. The method of claim 19, further comprising treating the subject for AD based on the presence of the one or more genetic variation.

36. The method of claim 19 further comprising detecting in the sample the presence of at least one APOE-ε4 allele.

37. The method of claim 36, wherein the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

38. A method for diagnosing or prognosing AD in a subject, comprising:
(a) contacting a sample from the subject with a reagent capable of detecting the presence or absence of a genetic variation in a gene selected from the genes encoding IL6R, NTF4 and UNC5C, or a gene product thereof; and
(b) determining the presence or absence of the genetic variation, wherein the presence of the genetic variation indicates that the subject is afflicted with, or at risk of developing, AD.

39. The method of claim 38, wherein the at least one genetic variation is a single nucleotide polymorphism (SNP), an allele, a haplotype, an insertion, or a deletion.

40. The method of claim 39, wherein the genetic variation is a SNP.

41. The method of claim 40 wherein the genetic variation is selected from the group consisting of a SNP that results in the amino acid substitution D358A in the amino acid sequence of IL6R (SEQ ID NO:1), a SNP that results in the amino acid substitution R206W in the amino acid sequence of NTF4 (SEQ ID NO:2), and a SNP that results in the amino acid substitution T835M in the amino acid sequence of UNC5C (SEQ ID NO:3).

42. The method of claim 34, wherein the genetic variation is selected from a ‘C’ allele at rs2228145, a ‘T’ allele at rs121918427, and a SNP that substitutes G for A in the codon encoding for the amino acid at position 835 of UNC5C (SEQ ID NO:3).

43. The method of claim 38 wherein the reagent is selected from an oligonucleotide, a DNA probe, an RNA probe, and a ribozyme.

44. The method of claim 43 wherein the reagent is labeled.

45. The method of claim 38 wherein the at least one genetic variation is an amino acid substitution, insertion or deletion in a protein selected from IL6R, NTF4 and UNC5C.

46. The method of claim 45 wherein the at least one genetic variation is an amino acid substitution selected from D358A in the amino acid sequence of IL6R (SEQ ID NO:1), R206W in the amino acid sequence of NTF4 (SEQ ID NO:2), and T835M in the amino acid sequence of UNC5C (SEQ ID NO:3).

47. The method of claim 45 wherein the reagent is an antibody that specifically binds to a protein comprising the genetic variation.

48. The method of claim 38, wherein the sample is selected from one of cerebrospinal fluid, blood, serum, sputum, saliva, mucosal scraping, tissue biopsy, lacrimal secretion, semen, or sweat.

49. The method of claim 38, further comprising treating the subject for AD based on the results of step (b).

50. The method of claim 38 further comprising detecting in the sample the presence of at least one APOE-ε4 allele.

51. The method of claim 50, wherein the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

52. The method of claim 38, further comprising subjecting the subject to one or more additional diagnostic tests for AD selected from the group consisting of screening for one or more additional genetic markers, administering a mental status exam, or subjecting the subject to imaging procedures.

53. The method of claim 38 further comprising analyzing the sample to detect the presence of at least one additional genetic marker that is an APOE modifier, wherein the at least one additional genetic marker is in a gene selected from the gene encoding IL6R, the gene encoding NTF4, the gene encoding UNC5C, and a gene listed in Table 3.

54. The method of claim 53 wherein the at least one additional genetic marker is a SNP that results in the amino acid substitution D358A in the amino acid sequence of IL6R (SEQ ID NO:1), a SNP that results in the amino acid substitution R206W in the amino acid sequence of NTF4 (SEQ ID NO:2), a SNP that results in the amino acid substitution T835W in the amino acid sequence of UNC5C (SEQ ID NO:3), or a SNP that is listed in Table 3.

55. A method of diagnosing or prognosing AD in a subject, comprising:
determining the presence or absence of a genetic variation in a gene selected from the genes encoding IL6R, NTF4 and UNC5C, or a gene product thereof, in a biological sample from a subject, wherein the presence of the genetic variation indicates that the subject is afflicted with, or at risk of developing, AD.

56. The method of claim 55, wherein the at least one genetic variation is a single nucleotide polymorphism (SNP), an allele, a haplotype, an insertion, or a deletion.

57. The method of claim 56, wherein the genetic variation is a SNP.

58. The method of claim 57 wherein the genetic variation is selected from SNP that results in the amino acid substitution D358A in the amino acid sequence of IL6R (SEQ ID NO:1), a SNP that results in the amino acid substitution R206W in the amino acid sequence of NTF4 (SEQ ID NO:2), and a SNP that results in the amino acid substitution T835M in the amino acid sequence of UNC5C (SEQ ID NO:3).

59. The method of claim 57, wherein the genetic variation is selected from a ‘C’ allele at rs2228145, a ‘T’ allele at rs121918427, and a SNP that substitutes G for A in the codon encoding for the amino acid at position 835 of UNC5C (SEQ ID NO:3).

60. The method of claim 55 wherein the presence of the one or more genetic variation is carried out by a process selected from the group consisting of direct sequencing, allele-specific probe hybridization, allele-specific primer extension, allele-specific amplification, allele-specific nucleotide incorporation, 5′ nuclease digestion, molecular beacon assay, oligonucleotide ligation assay, size analysis, and single-stranded conformation polymorphism.

61. The method of claim 60 wherein nucleic acids from the sample are amplified prior to determining the presence of the one or more genetic variation.

62. The method of claim 55 wherein the at least one genetic variation is an amino acid substitution, insertion or deletion in a protein selected from IL6R, NTF4 and UNC5C.

63. The method of claim 62 wherein the at least one genetic variation is an amino acid substitution selected from D358A in the amino acid sequence of IL6R (SEQ ID NO:1), R206W in the amino acid sequence of NTF4 (SEQ ID NO:2), and T835M in the amino acid sequence of UNC5C (SEQ ID NO:3).

64. The method of claim 55 wherein the presence of the one or more genetic variation is carried out by a process selected from electrophoresis, chromatography, mass spectroscopy, proteolytic digestion, protein sequencing, immunoaffinity assay, or a combination thereof.

65. The method of claim 64 wherein proteins from the sample are purified using antibodies or peptides that bind the proteins prior to determining the presence of the one or more genetic variation.

66. The method of claim 55, wherein the sample is selected from one of cerebrospinal fluid, blood, serum sputum, saliva, mucosal scraping, tissue biopsy, lacrimal secretion, semen, or sweat.

67. The method of claim 55, further comprising treating the subject for AD based on the presence of the one or more genetic variation.

68. The method of claim 55 further comprising detecting in the sample the presence of at least one APOE-ε4 allele.

69. The method of claim 68, wherein the presence of the at least one genetic variation together with the presence of at least one APOE-ε4 allele is indicative of an increased risk of earlier age of diagnosis of AD compared to a subject having at least one APOE-ε4 allele and lacking the presence of the at least one genetic marker.

70. The method of claim 55, further comprising subjecting the subject to one or more additional diagnostic tests for AD selected from the group consisting of screening for one or more additional genetic markers, administering a mental status exam, or subjecting the subject to imaging procedures.

71. The method of claim 55 further comprising analyzing the sample to detect the presence of at least one additional genetic marker that is an APOE modifier, wherein the at least one additional genetic marker is in a gene selected from the gene encoding IL6R, the gene encoding NTF4, the gene encoding UNC5C, and a gene listed in Table 3.

72. The method of claim 71 wherein the at least one additional genetic marker is a SNP that results in the amino acid substitution D358A in the amino acid sequence of IL6R (SEQ ID NO:1), a SNP that results in the amino acid substitution R206W in the amino acid sequence of NTF4 (SEQ ID NO:2), a SNP that results in the amino acid substitution T835W in the amino acid sequence of UNC5C (SEQ ID NO:3), or a SNP that is listed in Table 3.

73. A method of identifying a subject having an increased risk of earlier age of diagnosis of AD, comprising:
determining the presence or absence of a genetic variation in a gene selected from the genes encoding IL6R, NTF4 and UNC5C, or a gene product thereof, in a biological sample from a subject,
determining the presence or absence of at least one APOE-ε4 allele,
wherein the presence of the genetic variation and at least one APOE-ε4 allele indicates that the subject has an increased risk of earlier age of diagnosis of AD as compared to a subject lacking the presence of the genetic variation and at least one APOE-ε4 allele.

74. A method of aiding prognosis of a subphenotype of AD in a subject, the method comprising detecting in a biological sample derived from the subject the presence of a SNP that results in the amino acid substitution D358A in the amino acid sequence of IL6R (SEQ ID NO:1), wherein the subphenotype of AD is characterized at least in part by increased levels of soluble IL6R in a biological sample derived from the subject as compared to one or more control subjects.

75. A method of predicting the response of a subject to an AD therapeutic agent that targets IL6R, comprising detecting in a biological sample obtained from the subject a SNP that results in the amino acid substitution D358A in the amino acid sequence of IL6R (SEQ ID NO:1), wherein the presence of the SNP is indicative of a response to a therapeutic agent that targets IL6R.

76. The method of claim 75 wherein the therapeutic agent is an anti-IL6R antibody.

77. A method of aiding prognosis of a subphenotype of AD in a subject, the method comprising detecting in a biological sample derived from the subject the presence of a SNP that results in the amino acid substitution R206W in the amino acid sequence of NTF4 (SEQ ID NO:2), wherein the subphenotype of AD is characterized at least in part by decreased activation of TrkB in a biological sample derived from the subject as compared to one or more control subjects.

78. A method of predicting the response of a subject to an AD therapeutic agent that targets TrkB, comprising detecting in a biological sample obtained from the subject a SNP that results in the amino acid substitution R206W in the amino acid sequence of NTF4 (SEQ ID NO:2), wherein the presence of the SNP is indicative of a response to a therapeutic agent that targets TrkB.

79. The method of claim 78 wherein the therapeutic agent is a TrkB agonist.

80. A method of aiding prognosis of a subphenotype of AD in a subject, the method comprising detecting in a biological sample derived from the subject the presence of a SNP that results in the amino acid substitution T835M in the amino acid sequence of UNC5C (SEQ ID NO:3), wherein the subphenotype of AD is characterized at least in part by increased apoptotic activity of UNC5C in a biological sample derived from the subject as compared to one or more control subjects.

81. A method of predicting the response of a subject to an AD therapeutic agent that targets UNC5C, comprising detecting in a biological sample obtained from the subject a SNP that results in the amino acid substitution T835M in the amino acid sequence of UNC5C (SEQ ID NO:3), wherein the presence of the SNP is indicative of a response to a therapeutic agent that targets UNC5C.

82. The method of claim 81 wherein the therapeutic agent targets the UNC5C death domain.

83. A method of diagnosing or prognosing Alzheimer's Disease (AD) in a subject, comprising:
(a) contacting a sample from the subject with a reagent capable of detecting the presence or absence of one or more SNPs selected from the group consisting of a SNP that results in the amino acid substitution D358A in the amino acid sequence of IL6R (SEQ ID NO:1), a SNP that results in the amino acid substitution R206W in the amino acid sequence of NTF4, and a SNP that results in the amino acid substitution T835M in the amino acid sequence of UNC5C (SEQ ID NO:3), and
(b) analyzing the sample to detect the presence of said one or more SNPs, wherein the presence of the one or more SNPs in the sample indicates that the subject is afflicted with, or at risk of developing, AD.

84. The method of claim 85, further comprising detecting one or more SNPs selected from the SNPs listed in Table 3.

85. A kit for carrying out the method of claim 83, comprising at least one oligonucleotide detection reagent, wherein the oligonucleotide detection reagent distinguishes between each of at least two different alleles at the one or more SNP.

86. The kit of claim 85, wherein the detecting is carried out by a process selected from the group consisting of direct sequencing, allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

87. The kit of claim 85, wherein the oligonucleotide detection reagents are immobilized to a substrate.

88. The kit of claim 87, wherein the oligonucleotide detection reagents are arranged on an array.

89. A method of diagnosing or prognosing Alzheimer's Disease (AD) in a subject, comprising:
(a) contacting a sample from the subject with a reagent capable of detecting the presence or absence of one or more amino acid substitutions selected from the group consisting of the amino acid substitution D358A in the amino acid sequence of IL6R (SEQ ID NO:1), the amino acid substitution R206W in the amino acid sequence of NTF4, and the amino acid substitution T835M in the amino acid sequence of UNC5C (SEQ ID NO:3), and
(b) analyzing the sample to detect the presence of said one or more amino acid substitutions, wherein the presence of the one or more amino acid substitutions in the sample indicates that the subject is afflicted with, or at risk of developing, AD.

90. A kit for carrying out the method of claim 89, comprising at least one antibody detection reagent, wherein the antibody detection reagent distinguishes between each of at least two different amino acids at the one or more amino acid substitution.

91. A therapeutic target for the treatment of AD, wherein the therapeutic target is one or a combination of proteins encoded by the genes selected from IL6R, NTF4 and UNC5C.

92. A set of molecular probes for diagnosis or prognosing AD comprising at least two probes capable of detecting directly or indirectly at least two markers selected from the group comprising: a SNP that results in the amino acid substitution D358A in the amino acid sequence of IL6R (SEQ ID NO:1), a SNP that results in the amino acid substitution R206W in the amino acid sequence of NTF4, and a SNP that results in the amino acid substitution T835M in the amino acid sequence of UNC5C (SEQ ID NO:3), wherein said molecular probes are not associated with a microarray of greater than 1000 elements.

93. The set of molecular probes of claim 92, further comprising one or more probes capable of detecting directly or indirectly at least two markers selected from the SNPs listed in Table 3.

94. A method of screening for genetic variants having a detrimental or beneficial effect on the development of AD in subjects having at least one APOE-ε4 allele, the method comprising identifying a genetic variant that is present at increased or decreased frequency in subjects under 65 years of age, having AD, and having at least one APOE-ε4 allele, as compared to control subjects over 75 years of age, without AD, and having at least one APOE-ε4 allele, wherein increased frequency in subjects having AD as compared to control subjects indicates that the genetic variation is associated with a detrimental effect in subjects having at least one APOE-ε4 allele, and decreased frequency in subjects having AD as compared to control subjects indicates that the genetic variation is associated with a beneficial effect in subjects having at least one APOE-ε4 allele.

95. The method of claim 94 wherein the genetic variation is identified using a genome-wide association scan.

96. The method of claim 94 wherein the detrimental effect is increased risk of developing AD or a lower age of onset of AD.

97. The method of claim 94 wherein the beneficial effect is decreased risk of developing AD or a later age of onset of AD.

98. A method of screening for genetic variants having a detrimental or beneficial effect on the development of AD in subjects having at least one APOE-ε4 allele, the method comprising
(a) determining the genotype at one or more genetic locus of a plurality of subjects under 65 years of age, having AD, and having at least one APOE-ε4 allele;
(b) determining the genotype at one or more genetic locus of a plurality of control subjects over 75 years of age, without AD, and having at least one APOE-ε4 allele; and
(c) identifying a genetic variant that is present at increased or decreased frequency in subjects having AD as compared to control subjects, wherein increased frequency in subjects having AD as compared to control subjects indicates that the genetic variation is associated with a detrimental effect in subjects having at least one APOE-ε4 allele, and decreased frequency in subjects having AD as compared to control subjects indicates that the genetic variation is associated with a beneficial effect in subjects having at least one APOE-ε4 allele.


SEQUENCE LISTING (18 sequences)

SEQ ID NO: 1 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 2 - protein, 210 aa, Homo sapiens
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 3 - protein, 931 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIQLDAEACHILTENLSTYALVGHSTTKAAAKRLKLAIFGPLCCSSLEYSIRVYCLDDTQDALKEILHLERQMGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 4 - protein, 83 aa, Rattus norvegicus
HCTFTLERHSLASTEFTCKVCVRQVEGEGQIFQLHTTLAETPAGSLDALCSAPGNAATTQLGPYAFKIPLSIRQKICNSLDAP

SEQ ID NO: 5 - protein, 83 aa, Homo sapiens
HCTFTLERHSLASTELTCKICVRQVEGEGQIFQLHTTLAETPAGSLDTICSAPGSTVTTQLGPYAFKIPLSIRQKICNSLDAP

SEQ ID NO: 6 - protein, 83 aa, Mus musculus
HCTFTLERHSLASTEFTCKVCVRQVEGEGQIFQLHTTLAETPAGSLDALCSAPGNAITTQLGPYAFKIPLSIRQKICSSLDAP

SEQ ID NO: 7 - protein, 82 aa, Xenopus Laevis
HCTFTLERYSLAATELTCKICVRQVEGEGQIFQLHTLLEENVKSFDPFCSQAENSVTTHLGPYAFKIPFSIRQKICNSLDAP

SEQ ID NO: 8 - protein, 82 aa, Rattus norvegicus
HCTFTLERINASTSDLACKVWVWQVEGDGQSFNINFNITKDTRFAELLALESEGGVPALVGPSAFKIPFLIRQKIIASLDPP

SEQ ID NO: 9 - protein, 82 aa, Homo sapiens
HCTFTLERVSPSTSDLACKLWVWQVEGDGQSFSINFNITKDTRFAELLALESEAGVPALVGPSAFKIPFLIRQKIISSLDPP

SEQ ID NO: 10 - protein, 82 aa, Mus musculus
HCTFTLERVNASTSDLACKVWVWQVEGDGQSFNINFNITKDTRFAEMLALESEGGVPALVGPSAFKIPFLIRQKIITSLDPP

SEQ ID NO: 11 - protein, 82 aa, Rattus norvegicus
HCTFTLERLSLNTVELVCKLCVRQVEGEGQIFQLNCTVSEEPTGIDLPLLDPASTITTVTGPSAFSIPLPIRQKLCSSLDAP

SEQ ID NO: 12 - protein, 82 aa, Homo sapiens
HCTFTLERFSLNTVELVCKLCVRQVEGEGQIFQLNCTVSEEPTGIFLPLLDPANTITTVTGPSAFSIPLPIRQKLCSSLDAP

SEQ ID NO: 13 - protein, 82 aa, Mus musculus; identical to SEQ ID NO: 11

SEQ ID NO: 14 - protein, 82 aa, Gallus gallus
HCTFTLERFSLNTLELVCKLCVRQVEGEGQIFQLNCSVSEEPTGIDYPIMDSAGSITTIVGPNAFSIPLPIRQKLCSSLDAP

SEQ ID NO: 15 - protein, 82 aa, Homo sapiens
HCAFSLERYTPTTTQLSCKICIRQLKGHEQILQVQTSILESERETITFFAQEDSTFPAQTGPKAFKIPYSIRQRICATFDTP

SEQ ID NO: 16 - protein, 82 aa, Mus musculus; identical to SEQ ID NO: 15

SEQ ID NO: 17 - protein, 76 aa, Caenorhabditis elegans
HCSLKFRPKEINGSQFSTRVIVYQKASSTEPMVMEVSNEPELYDATSEEREKGSVCVEFRLPFGVKDELARLLDMP

SEQ ID NO: 18 - protein, 86 aa, Camelus dromedarius
HCEFSLRRQDQNSLCVDFGQGSEDDYYTFNIPAHSMSGSAEELASTTNTTISIDRQGNYVNESCVMDFVQLPHATKRLICGALDPP